(19) United States
(12) Patent Application Publication
(10) Pub. No.: US 2006/0019890 A1
(43) Pub. Date: Jan. 26, 2006

(54) Method for treating cardiac remodeling following myocardial injury

(76) Inventors: Ann M. Kapoun, Mountain View, CA (US); George F. Schreiner, Los Altos, CA (US); Faquan Liang, San Francisco, CA (US); Zhihe Li, Foster City, CA (US)

Correspondence Address: Samuel M. Kais;Scios Inc., 6500 Paseo Padre Parkway, Fremont, CA, 94555, US

(21) Appl. No.: 11/038,826

(22) Filed: Jan. 18, 2005

Related U.S. Application Data
(60) Provisional application No. 60/537,221, filed on Jan. 15, 2004.

(51) Int. Cl. A61K 38/17

(52) U.S. Cl. 514/12

(57) ABSTRACT

The invention concerns methods for treating cardiac remodeling in a subject who has undergone myocardial injury, said method comprising the administration of natriuretic peptide to said subject. Preferably the natriuretic peptide is brain natriuretic peptide. The invention also concerns methods for treating structural heart disorders arising from myocardial injury, said method comprising the administration of a natriuretic peptide to a patient in need thereof.

This application claims priority to U.S. provisional application Ser. No. 60/537,221. The 60/537,221 provisional application is herein incorporated by reference in its entirety.

### FIELD OF THE INVENTION

The present invention concerns methods of treatment using one or more natriuretic peptides or derivatives thereof. More specifically, the invention concerns methods of treating or preventing cardiac dysfunction in a subject after said subject has undergone myocardial injury.

### BACKGROUND

Myocardial infarction is a major cause of significant disability and death in the United States and in many other countries around the world, and accounts for approximately ⅔ of all heart failure. Hunt et al, AMERICAN COLLEGE OF CARDIOLOGY/AMERICAN Heart Association. ACC/AHA guidelines for the evaluation and management of chronic heart failure in the adult: executive summary. A report of the American College of Cardiology/American Heart Association Task Force on Practice Guidelines (Committee to revise the 1995 Guidelines for the Evaluation and Management of Heart Failure). Journal of the American College of Cardiology 2001; 38: 2101-2113. Several disease-initiating events (e.g. myocardial infarction, untreated hypertension, congenital mutations of contractile proteins) can result in a common heart disease phenotype that consists of dilation of the cardiac chambers, resulting in reduction in contractile function (i.e., a decrease in the fraction of total blood ejected from each chamber during systole) that leads to the clinical syndrome of heart failure. This phenotype generally involves a compensatory aspect that results from myocardial infarction when the normal compensatory hypertrophy of surviving, non-infarcted myocardium is insufficient. Often this compensatory mechanism is a result of the profibrotic response associated with cardiac injury.

Available therapies for heart dysfunction are insufficient, and new methods of treatment are needed. The heart responds to infarction by hypertrophy of surviving cardiac muscle in an attempt to maintain normal contraction. However, when the hypertrophy is insufficient to compensate, cardiac remodeling and reduced cardiac function result, leading to heart failure and death. Despite important advances in medical therapies for preventing cardiac dysfunction and heart failure after myocardial infarction, these problems remain a significant unsolved public health problem.

No pharmacological therapy for post MI cardiac remodeling is curative or satisfactory, and many patients die or, in selected cases, undergo heart transplantation. Presently available pharmacological therapies for reducing cardiac dysfunction and reducing mortality in patients with heart failure fall into three main categories: angiotensin-converting enzyme (ACE) inhibitors, beta adrenergic receptor (OAR) antagonists, and aldosterone antagonists. Despite reducing mortality, patients treated with these medicines remain at significantly increased rislc for death compared to age-matched control patients without heart failure. ACE inhibitors, βAR antagonists and (at least one type of) aldosterone receptor antagonist can significantly reduce the incidence and extent of cardiac dysfunction and heart failure after myocardial infarction.

ACE inhibitors are associated with cough in 10% of patients and can result in renal failure in the setting of bilateral renal artery stenosis or other severe kidney disease. βAR antagonists are associated with impotence and depression, and are contraindicated in patients with asthma; furthermore, patients may develop worsened heart failure, hypotension, bradycardia, heart block, and fatigue with initiation of βAR antagonists. Aldosterone receptor antagonism causes significant hyperkalemia and painful gynecomastia in 10% of male patients. Agents without a demonstrated mortality benefit are also associated with problems; most notable is the consistent finding that many cardiac stimulants improve symptoms, but actually increase mortality, likely by triggering lethal cardiac arrhythmias. In summary, presently available pharmacological therapies are ineffective and are limited by significant unwanted side effects, and so development of new therapies with improved efficacy and less severe side effects is an important public health goal.

### SUMMARY OF THE INVENTION

The present invention is directed to the use of natriuretic peptides for the prevention and/or treatment of cardiac remodeling in a subject that has undergone myocardial injury. In a preferred embodiment, the natriuretic peptide(s) comprise brain natriuretic peptide (BNP), also known as nesiritide. In another embodiment, the invention is directed to the treatment of cardiac dysfunction, said treatment comprising the administration of a therapeutically effective amount of natriuretic peptide to a subject that has undergone myocardial injury.

In another related embodiment, the invention is directed to a method of alleviating or reversing the effect of TGFβ mediated cell activation in cardiac tissue on the expression of one or more genes associated with fibrosis, comprising contacting one or more cells or tissues in which the expression of said genes is altered as a result of TGFβ mediated activation, with BNP. In another related embodiment, the targeted gene(s) associated with fibrosis are selected from the group consisting essentially of Collagen1, Collagent 3, Fibronectin, CTGF, PAI-1, and TIMP3.

In another embodiment, the invention is directed to a method of inhibiting the production of Collagen 1, Collagen 3 or Fibronectin proteins by the administration of a therapeutically effective amount of BNP to a subject in need thereof.

In another related embodiment, the invention is directed to a method of inhibiting TGFβ mediated myofibroblast conversion by administration of a therapeutically effective amount of BNP to a mammalian subject in need thereof.

In another related embodiment, the invention is directed to a method of alleviating or reversing the effect of TGFβ mediated cell activation in cardiac tissue on the expression of one or more genes associated with cell proliferation, comprising contacting one or more cells or tissues in which the expression of said genes is altered as a result of TGFβ mediated activation, with BNP. In another related embodiment, the targeted gene(s) associated with cell proliferation are selected from the group consisting essentially of PDGFA, IGF1, FGF18, and IGFBP10.

In another related embodiment, the invention is directed to a method of alleviating or reversing the effect of TGFβ mediated cell activation in cardiac tissue on the expression of one or more genes associated with inflammation, comprising contacting one or more cells or tissues in which the expression of said genes is altered as a result of TGFβ mediated activation, with BNP. In another related embodiment, the targeted gene(s) associated with inflammation are selected from the group comprise COX1, IL6, TNFα-inducted protein 6, TNF superfamily, member 4.

### BRIEF DESCRIPTION OF THE DRAWINGS

FIG. 1. Gene expression changes induced by TGFβ and BNP in human cardiac fibroblasts at 24 and 48 h. Histograms show the number of gene expression changes that were up-regulated and down-regulated by TGFβ and BNP treatment. Hybridizations using fluorescently-labeled cDNA probes compare untreated (control) to TGFβ-treated cells and control to BNP-treated cells. See Experimental for details related to the gene expression values. Histogram bars: 24 h (white) and 48 h (black).

FIG. 2. Effects of BNP on TGFβ-induced gene expression in human cardiac fibroblasts. Hybridizations using fluorescently-labeled cDNA probes compare TGFβ-treated to TGFβ BNP-treated cells at 24 and 48 h. Strong and weak effects represent 1.8- and 1.5-fold gene expression levels, respectively. See Experimental for details related to statistical significance. Histogram bars: no effect (white), weak effect (grey), and strong effect (black).

FIG. 3. Gene expression patterns in TGFβ-treated human cardiac fibroblasts. Data was generated using the hierarchical clustering algorithm contained in Spotfire™ software. Each row represents one of 524 genes, and each column represents the results from duplicate hybridizations: (A) control vs. TGFβ, 24 h; (B) control vs. TGFβ, 48 h; (C) TGFβ vs. TGFβ+BNP 24 h; (D) TGFβ vs. TGFβ+BNP 48 h; (E) control vs. BNP 24 h; and (F) control vs. BNP 48 h. Normalized data values depicted in shades of red and green represent elevated and repressed expression, respectively. See Table 2 in Experimental section for gene identities and expression values.

FIG. 4. Gene expression clusters in human cardiac fibroblasts: (A) fibrosis and ECM, (B) cell proliferation, and (C) inflammation. See FIG. 4 legend for descriptions of the hybridizations and gene expression color codes.

FIG. 5. Effects of BNP on TGFβ-induced Collagen 1 (A and B) and Fibronectin (C and D) mRNA and protein levels in cultured human cardiac fibroblasts. Histograms show control cells (white), cells treated with BNP (gray), cells treated with TGFβblack), and cells co-treated with BNP and TGFβ(hatched). (A and C) Real-time RT-PCR expression levels were normalized to 18S rRNA and plotted relative to the level in the 6 h control cells. Error bars reflect duplicate biological replicates; real-time RT-PCR reactions were performed in triplicate. (B and D) Western blot analyses are presented as mean±SD from three separate experiments; *p<0.01 vs. control; **p<0.01 vs. TGFβ.

FIG. 6. Effects of BNP on TGFβ-induced fibrotic and inflammatory genes. Real-time RT-PCR expression levels were normalized to 18S rRNA and plotted relative to the level in the 6 h control cells. See FIG. 5 for key to histogram bar labels and error bars.

FIG. 7. Effect of PKG and MEK inhibitors on BNP-dependent inhibition of TGFβ signaling in human cardiac fibroblasts. (A) Western analysis of ERK phosphorylation. Cells were treated with BNP (0.5 μmol/L) in the presence or absence of KT5823 (1 μmol/L) or U0126 (10 μmol/L) for 15 min. (B) Western blot and (C) real-time RT-PCR analysis to detect Collagen 1 expression. Cells were treated with 5 ng/ml TGFβ and/or BNP (100 nmol/L, three times daily) in the presence or absence of KT5823 (1 μmol/L), U0126 (0.1-10 μmol/L) or PD98059 (10 μmol/L) for 48 h. Control (C); KT5823 (KT); U0126 (U); TGFβ (TGF).

FIG. 8. Summary of BNP effects on gene expression in TGFβ-stimulated human cardiac fibroblasts.

FIG. 9. Effects of BNP on TGFβ-stimulated fibroblast proliferation. Histograms show fold induction of BrdU labeled cells treated with TGFβ alone, BNP alone or co-treated with BNP and TGFβ. Cells were co-treated with BNP and TGFβ for 24 h, then labeled with BrdU and cultured for an additional 24 h. Pooled data represent the mean±SD from three individual experiments: *p<0.01 vs. the control; **p<0.05 vs. TGFβ.

FIG. 10. Changes in plasma aldosterone level. The increased plasma aldosterone level by L-NAME/AngII was reduced by BNP (p<0.05, n=7)

FIG. 11. Changes in heart/body weight ratio. BNP abolished L-NAME/AngII-induced increase in heart/body weight ratio (p<0.01, n=12)

FIG. 12. Real time RT-PCR results. Expression of mRNA of collagen I (A), collagen III (B) and fibronectin (C) in the heart. BNP abolished the fibrotic genes that enhanced by L-NAME plus Angiotensin II (p<0.01 in all cases).

FIG. 13. Cardiac function parameters including heart rate (A), stroke volume (B), ejection fraction (C), cardiac output (D), stroke work (E), maximum dP/dt (F), minimum dP/fy (G), and arterial elastance (H). L-NAME/AngII induced deterioration of cardiac function. Administration of BNP significantly improved cardiac function as judged by increases in stroke volume, ejection fraction, cardiac output, stroke work and decrease in arterial elastance (p<0.001, n=8). BNP also increased maximum dP/dt (p<0.05) and minimum dP/dt. BNP had no effect on heart rate.

### DETAILED DESCRIPTION

A. Definitions

As used herein, any reference to “reversing the effect of TGF-β-mediated cell activation on the expression of a gene associated with fibrosis” means partial or complete reversal the effect of TGF-β-mediated cell activation of that gene, relative to a normal sample of the same cell or tissue type. It is emphasized that total reversal (i.e. total return to the normal expression level) is not required, although is advantageous, under this definition.

The term “cardiac remodeling” generally refers to the compensatory or pathological response following myocardial injury. Cardiac remodeling is viewed as a key determinant of the clinical outcome in heart disorders. It is characterized by a structural rearrangement of the cardiac chamber wall that involves cardiomyocyte hypertrophy, fibroblast proliferation, and increased deposition of extracellular matrix (ECM) proteins. Cardiac fibrosis is a major aspect of the pathology typically seen in the failing heart. The proliferation of interstitial fibroblasts and increased deposition of extracellular matrix components results in myocardial stiffness and diastolic dysfunction, which ultimately leads to heart failure. A number of neurohumoral or growth factors have been implicated in the development of cardiac fibrosis. These include angiotensin II (AII), endothelin-1 (ET-1), cardiotrophin-1 (CT-1), norepinephrine (NE), aldosterone, FGF2, PDGF, and transforming growth factored (TGFβ). TGFβ expression is also stimulated by AII and ET-1 in cardiac myocytes and fibroblasts, further supporting its involvement in cardiac fibrosis.

The term “cardiac dysfunction” refers to the pathological decline in cardiac performance following myocardial injury. Cardiac dysfunction may be manifested through one or more parameters or indicia including changes to stroke volume, ejection fraction, end diastolic fraction, stroke work, arterial elastance, or an increase in heart weight to body weight ratio.

The terms “differentially expressed gene,” “differential gene expression” and their synonyms, which are used interchangeably, refer to a gene whose expression is activated to a higher or lower level in a test sample relative to its expression in a normal or control sample. For the purpose of this invention, “differential gene expression” is considered to be present when there is at least an about 2.5-fold, preferably at least about 4-fold, more preferably at least about 6-fold, most preferably at least about 10-fold difference between the expression of a given gene in normal and test samples.

“Myocardial injury” means injury to the heart. It may arise from myocardial infarction, cardiac ischemia, cardiotoxic compounds and the like. Myocardial injury may be either an acute or nonacute injury in terms of clinical pathology. In any case it involves damage to cardiac tissue and typically results in a structural or compensatory response.

As used herein, “natriuretic peptides” means a composition that includes one or more of an Atrial natriuretic peptide (ANP), a Brain natriuretic peptide (BNP), or a C-type natriuretic peptide (CNP). It is contemplated that analogues and variants of these peptides be included in the definition. Examples of such include anaritide (ANP analogue of different length) or combinations of natriuretic peptide including but not limited to ANP/BNP, ANP/CNP, an BNP/CNP variants. Preferably, natriuretic peptide means BNP (nesiritide).

The terms “treating” or “alleviating” refer to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In the treatment of a fibroproliferative disease, a therapeutic agent may directly decrease the pathology of the disease, or render the disease more susceptible to treatment by other therapeutic agents.

The term “subject” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the subject is human.

Administration “in combination with” one or more further therapeutic agents includes simultaneous (concurrent) and consecutive administration in any order.

A “therapeutically effective amount”, in reference to the treatment of cardiac or renal fibrosis, e.g. when inhibitors of the present invention are used, refers to an amount capable of invoking one or more of the following effects: (1) inhibition (i.e., reduction, slowing down or complete stopping) of the development or progression of fibrosis and/or sclerosis; (2) inhibition (i.e., reduction, slowing down or complete stopping) of consequences of or complications resulting from such fibrosis and/or sclerosis; and (3) relief, to some extent, of one or more symptoms associated with the fibrosis and/or sclerosis, or symptoms of consequences of or complications resulting from such fibrosis and/or sclerosis.

B. Modes of Carrying out the Invention

Natriuretic peptides comprise a family of vasoactive hormones that play important roles in the regulation of cardiovascular and renal homeostasis. Atrial natriuretic peptide (ANP) and brain natriuretic peptide (BNP) are predominantly produced in the heart and exert vasorelaxant, natriuretic, and anti-growth activities. Binding of ANP and BNP to type-A natriuretic peptide receptor (NPRA) leads to the generation of cyclic guanosine monophosphate (cGMP), which mediates most biological effects of the peptides. Mice lacking NPRA exhibit cardiac hypertrophy, fibrosis, hypertension and increased expression of fibrotic genes including TGFβ1, TGFβ3 and Collagen 1. Furthermore, targeted disruption of the BNP gene in mice results in cardiac fibrosis and enhanced fibrotic response to ventricular pressure overload, suggesting that BNP is involved in cardiac remodeling.

TGFβ mediates fibrosis by modulating fibroblast proliferation and ECM production, particularly of collagen and fibronectin. TGFβ also promotes the phenotypic transformation of fibroblasts into myofibroblasts characterized by expression of α-smooth muscle actin. Studies have demonstrated that increased myocardial TGFβ expression is associated with cardiac hypertrophy and fibrosis. Moreover, functional blockade of TGFβ prevents myocardial fibrosis and diastolic dysfunction in pressure overloaded rats, indicating that TGFβ has a crucial role in the process of myocardial remodeling, particularly in cardiac fibrosis. However, the implication of natriuretic peptide(s) in this process has not been previously explored.

The present invention is directed to the treatment or prevention of cardiac remodeling following myocardial injury. In a preferred embodiment, the myocardial injury comprises an acute myocardial infarction. Preferably the administration of natriuretic peptide occurs as soon as possible after the injury event.

In another embodiment, the invention involves the treatment of cardiac dysfunction in a subject in need thereof comprising the administration of a natriuretic peptide to a subject in need thereof wherein said administration occurs after said subject has undergone myocardial injury.

The manner of administration and formulation of the natriuretic peptide(s) useful in the invention will depend on the nature of the condition, the severity of the condition, the particular subject to be treated, and the judgment of the practitioner; formulation will depend on mode of administration. The peptides of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The peptides useful in the invention may also be administered through suppositories or other transmucosal vehicles. Typically, such formulations will include excipients that facilitate the passage of the compound through the mucosa such as pharmaceutically acceptable detergents.

The peptides may also be administered by injection, including intravenous, intramuscular, subcutaneous, intrarticular or intraperitoneal injection. Preferably the natriuretic peptide(s) are administered intravenously. Typical formulations for such use are liquid formulations in isotonic vehicles such as Hank's solution or Ringer's solution.

Alternative formulations include aerosol inhalants, nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the peptide(s) of the invention will depend on a number of factors which will vary from patient to patient. The dose regimen will vary, depending on the conditions being treated and the judgment of the practitioner. Further information regarding related formulations and dosages for brain natriuretic peptide can be found in the package insert or the latest version of Physicians Desk Reference (PDR) for nesiritide or the Natrecor® product.

It should be noted that the peptides useful for the invention can be administered as individual active ingredients, or as mixtures of several different compounds. In addition, the peptide(s) can be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system or genes associated with the development or progression of fibrotic diseases, and small molecule inhibitors of cell division, protein synthesis, or mRNA transcription or translation, or inhibitors of immune cell differentiation or activation.

As implicated above, although the peptide(s) of the invention may be used in humans, they are also available for veterinary use in treating non-human mammalian subjects.

Further details of the invention will be apparent from the Experimental section as provided below.

### EXPERIMENTAL

In vitro

Cell Culture

Two lots of primary human cardiac fibroblasts, derived from an 18-year old Caucasian male (lot 1) and a 56-year old Caucasian male (lot 2), were provided by Cambrex Bio Science (Walkersville, Md.). Cells stained positive for α-smooth muscle actin and vimenfin antibodies corroborating their identity as cardiac fibroblasts and myofibroblasts. Both lots were used for the real-time RT-PCR studies; lot 1 was used for the microarray analysis. Cells at passage 3-5 were cultured in FGM containing 15% FBS. At confluence, cells were split and cultured in 6-well plates for 24 h. Cells were changed to serum-free medium and treated with human BNP (American Peptide Company, Sunnyvale, Calif.) in the presence or absence of 5 ng/ml of TGFβ (R&D systems, Minneapolis, Minn.) for 6, 24 and 48 h. BNP and/or TGFβ-treated cells were also incubated in the presence of cGMP-dependent protein kinase (PKG) inhibitor KT5823 (1 μmol/L, Calbiochem, San Diego, Calif.), MAP kinase kinase (MEK) inhibitor U0126 (0.1-10 μmol/L, Sigma, St. Louis, Mo.) or PD98059 (10 μmol/L, Sigma) for 48 h. BNP (100 nmol/L) was added into the medium three times a day, such that the total calculated concentrations of exogenous BNP were 200 nmol/L, 600 nmol/L, and 900 nmol/L at 6, 24, and 48 h, respectively. This dosing protocol was necessary to maintain the levels of BNP in culture, since two distinct clearance pathways are responsible for the rapid degradation of natriuretic peptides. Without this treatment regime, it was found that BNP was significantly degraded in the cardiac fibroblasts; 50% of added BNP was metabolized within 24 h as measured by immunoreactive assays and cGMP stimulation cell bioassays.

Intracellular cGMP Assay

Cells were cultured in 6-well plates for 24 h, then changed to serum-free medium, and pre-incubated with 0.1 mmol/L of 3-isobutyl-1-methylxanthine (IBMX) for 1 h before treating with 10⁻⁹-10⁻⁶ mol/L of BNP for 10 min. The medium was aspirated and 0.5 ml of cold PBS was added into each well. Cells were scraped and mixed with 2 volumes of cold ethanol by vortex. After a 5 min room temperature incubation, the precipitate was removed by centrifugation at 1500×g for 10 min. The supernatant was dried by vacuum centrifugation, and levels of cGMP were measured using the cyclic GMP EIA kit (Cayman Chemical, Ann Arbor, Mich.).

BrdU incorporation

Cells were placed in 96-well plates and cultured for 24 h before changing to serum-free medium. Cells were treated with BNP (100 nmol/L, three times a day) in the presence or absence of 5 ng/ml of TGF-β for 24 h. Subsequently, 10 μmol/L of 5-bromo-2′-deoxyuridine (BrdU) was added to the cells, and they were cultured for an additional 24 h. BrdU incorporation was detected using the Cell Proliferation ELISA kit (Roche, Indianapolis, Ind.). Data was analyzed by ANOVA using the Newman-Keuls test to assess significance.

cDNA Microarray

Gene expression profiles were determined from cDNA microarrays containing 8,600 elements derived from clones isolated from normalized cDNA libraries or purchased from ResGen (Invitrogen Life Technologies, Carlsbad, Calif.). DNA for spotting was generated by PCR amplification using 5′amino-modified primers (BD Biosciences Clontech, Palo Alto, Calif.) derived from flanking vector sequences. Amplified DNA was purified in a 96-well format using Qiagen's Qiaquick columns (Valencia, Calif.) according to the manufacturer's recommendations. Samples were eluted in Milli-Q purified water, dried to completion and resuspended in 7 μl of 3×SSC. A fluorescent assay using PicoGreen (Molecular Probes, Eugene, Oreg.) was randomly performed on 12% of the PCR products to determine the average yield after purification; yields were ˜1.5 μg of DNA which corresponds to a concentration of 214 μg/ml. Purified DNA was arrayed from 384-well microtiter plates onto lysine-coated glass slides using an OmniGrid II microarrayer (GeneMachines, San Carlos, Calif.). After printing, DNA was cross-linked to the glass with 65 mjoules UV irradiation and reactive amines were blocked by treatment with succinic anhydride.

mRNA Isolation, Labeling, and Hybridizations

Total RNA was extracted from cells using Qiagen's RNeasy kit; two wells from a 6 well plate were pooled to yield a total of 4×10⁵ cells per treatment. RNA was amplified using a modified Eberwine protocol⁵ that incorporated a polyA tail into the amplified RNA. Fluorescently-labeled cDNA probes were generated by reverse transcription of 4 μg of RNA with SuperScript II (Invitrogen Life Technologies, Carlsbad, Calif.) using anchored dT primers in the presence of Cy3 or Cy5 dUTP (Amersham, Piscataway, N.J.). Labeled cDNA probe pairs were precipitated with ethanol and purified using Qiaquick columns. Twenty μg each of poly(A) DNA, yeast tRNA, and human Cot1 DNA (Applied Genetics, Melbourne, Fla.) was added to the eluant. The samples were dried to completion and resuspended in 12.5 μl 3×SSC, 0.1% SDS. Probes were heated to 95° C. for 5 minutes, applied to the arrays under a 22 mm² cover slip and allowed to hybridize for at least 16 h at 65° C. The arrays were washed at 55° C. for 10 minutes in 2×SSC, 0.1% SDS, followed by two washes at room temperature in 1×SSC (10 min) and 0.2×SSC (15 min). Hybridization of each fluorophore was quantified using an Axon GenePix 4000A scanner.

Microarray Data Analysis

Differential expression values were expressed as the ratio of the median of background-subtracted fluorescent intensity of the experimental RNA to the median of background-subtracted fluorescent intensity of the control RNA. For ratios greater than or equal to 1.0, the ratio was expressed as a positive value. For ratios less than 1.0, the ratio was expressed as the negative reciprocal (i.e., a ratio of 0.5=−2.0). Median ratios were normalized to 1.0 using two pools of 3000 randomly chosen cDNAs in each pool. Six replicates of each of the two pools were printed in 4 evenly distributed blocks of the array. Expression data was rejected if neither channel produced a signal of at least 2.0-fold over background. Differential expression ratios were determined as the mean of the two values from dye-swapped duplicates.

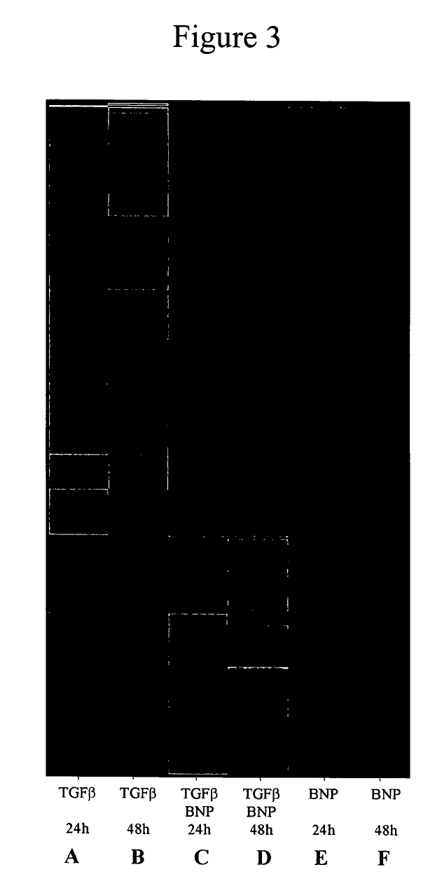

A statistically significant differential expression threshold value was empirically determined according to the method of Yang et al.⁵³ Seven independent self-self-hybridizations were performed in which the same RNA sample was labeled with Cy3 dUTP and Cy5 dUTP and hybridized to arrays containing 8,448 elements. Only elements that gave a signal greater than 2.0-fold over background in at least one of the dyes were considered in the analysis. Expression ratios were converted to log₍₂₎ and normalized to a mean=0. Combining data from all hybridizations, the 3 standard deviation limit was equivalent to a 1.48 fold change (+/−0.563 log₍₂₎). Of the 45,633 elements analyzed, 0.85% fell outside this threshold. Therefore, at this standard deviation limit, genes with fold changes greater than 1.48 can be considered differentially expressed at a 99% confidence level for any given hybridization. The percentage of elements that reproducibly fell outside the 3 standard deviation limit between any two duplicates of the seven self-self hybridizations was determined by comparing all 21 pair-wise combinations. An average of 18.9 elements +/−15.6 per hybridization duplicated at a fold change of 1.5, corresponding to a false positive rate of 0.29%. At a fold change of 1.8, an average of 0.71 elements +/−0.97 duplicated, corresponding to a false positive rate of 0.01%. A 1.8-fold threshold value was used to identify differentially expressed genes, except in FIG. 3, a 1.5-fold threshold value was used to designate “weak effects”.

Real-Time RT-PCR

Real-time RT-PCR¹⁸ was performed in a two-step manner. cDNA synthesis and real-time detection were carried out in a PTC-100™ Thermal Cycler (MJ Research Inc, Waltham, Mass.) and an ABI Prism™ 7700 Sequence Detection System (Applied Biosystems, Foster City, Calif.), respectively. Random hexamers (Qiagen, Valencia, Calif.) were used to generate cDNA from 200 ng RNA as described in Applied Biosystems User Bulletin #2. TaqMan™ PCR Core Reagent Kit or TaqMan™ Universal PCR Master Mix (Applied Biosystems) were used in subsequent PCR reactions according to the manufacturer's protocols. Relative quantitation of gene expression was performed using the relative standard curve method. All real-time RT-PCR reactions were performed in triplicate.

Sequence specific primers and probes were designed using Primer Express Version 2 software (Applied Biosystems). Sequences of primers and probes can be found in Table 1 below. Expression levels were normalized to 18S rRNA. The selection of 18S rRNA as an endogenous control was based on an evaluation of the ΔC_{T} levels (Applied Biosystems document # 4308134C) of 6 “housekeeping” genes: Cyclophilin A, 18S, GAPDH, β-actin, β-Glucuronidase, and Hypoxanthine Guanine Phosphoribosyl Transferase. The ΔC_{T} levels of 18S did not differ significantly between treatment conditions; thus, they were expressed at constant levels between samples.

TABLE 1

Real-time PCR primers and probes.
Western blot analysis

| | | | |
|---|---|---|---|
| Gene | Forward | Probe | Reverse |

| | | | |
|---|---|---|---|
| 18S | 5′-GCCGCTAGACGTGAAATTCTTG- | 5′-6FAM-AGCGGCGCAAGACGGACCAG-TAMRA-3′ | 5′-CATTCTTGGCAAATGCTTTCG- |
| | 3′ | | 3′ |
| Collagen1 | 5′-GGAATTGGGCTTCGACGTT-3′ | 5′-6FAM-TCTGCTTGCTGTAAACTCCCTGCATCCC- | 5′-TTCAGTTTGGGTTGCTTGTCTGT |
| | | TAMRA-3′ | -3′ |
| Fibronectin | 5′-AGATCTACCTGTACACCTTGAAT | 5′-6FAM-TGTCGTCATGGACGCCTCCA-TAMRA-3′ | 5′-CATGATACCAGCAAGGAATTGG- |
| | GACA-3′ | | 3′ |
| TIMP3 | 5′-TGTGTCATGTGAGGCTGTAATAT | 5′-6FAM-CACATCCCGCCATTTTGCTGAATCAA- | 5′-GGCTAGAAGTATTTTGCTCTCCA |
| | GTG-3′ | TAMRA-3′ | TTC-3′ |
| PAI-1 | 5′-GGCTGACTTCACGAGTCTTTCA- | 5′-6FAM-ACCAGAGGCTCTCGACGTCCCGG- | 5′-GTTCACCTCGATCTTCACTTTCT |
| | 3′ | TAMRA-3′ | G-3′ |
| CTGF | 5′-TGTGTGAGGAGCGCAAGGA-3′ | 5′-6FAM-CTGCCCTCGCGGCTTACCGA-TAMRA-3′ | 5′-TAGTTGGGTCTGGGCCAAAC-3′ |
| IL11 | 5′-AGAACAGCGAATTAAATGTGTCA | 5′-6FAM-AGACAAATGGCCCTCAAGTGGA- | 5′-CCCAGTTACGCAAGCATCCA-3′ |
| | TACA-3′ | TAMRA-3′ | |
| COX2 | 5′-GCTCAAACATGATGTTTGCATTC | 5′-6FAM-TTGCCCAGCACTTCAGGCATCAG- | 5′-GCCCTCGCTTATGATCTGTCTT- |
| | -3′ | TAMRA-3′ | 3′ |
| IL6 | 5′-ATGTAGCATGGCCACCTCAGAT- | 5′-6FAM-TGGTCAGAAACCTGTCCACTGGGCA- | 5′-TAACGCTCATACTTTTAGTTCTG |
| | 3′ | TAMRA-3′ | CATAGA-3′ |
| a-smooth | 5′-CCCCAGAGACCCTGTTGCA3′ | 5′6FAM-GCCAGCAGACTCCATGCCGA-TAMRA-3′ | 5′-TGATGCTGTTGTAGGTGGTTTCA |
| muscle actin | | | -3′ |

Cells were cultured in 6-well plates and treated with BNP (100 nM, three times daily) in the presence or absence of 5 ng/ml TGFβ for 48 h. Lysis was induced with 0.2 ml of buffer containing 20 mM Tris-HCL, pH 7.9, 137 mM NaCl, 1% Triton X-100, 5 mM EDTA, 10 mM NaF, 1 mM β-glycerophosphate, and protease inhibitor cocktail. The protein concentration of each lysate was measured using coomassie protein reagent from PIERCE. Twenty μg of protein from each sample was loaded and electrophoresed on 4-12% gradient polyacrylamide gels and electrophoretically transferred to nitrocellulose membranes (Invitrogen, San Diego, Calif.). The membranes were incubated with rabbit anti-human Collagen 1 antibody (Cortex Biochem, San Leandro, Calif.), HRP-conjugated anti-human Fibronectin antibody, or goat anti-Actin antibody (Santa Cruz Biotehnology, Santa Cruz, Calif.) in TBST buffer containing 20 mM Tris-HCl, pH 7.5, 150 mM NaCl, 0.1% Tween-20, and 5% nonfat dried milk at 4° C. for ˜16 h. For ERK phosphorylation, cells were treated with 0.5 μmol/L BNP in the presence of 1 μmol/L KT5823 or 10 μmol/L U0126 for 15 min; the membranes were incubated with rabbit anti-human phospho-ERK ½ antibody or rabbit anti-human ERK ½ antibody (Cell Signaling, Beverly, Mass.). For secondary antibody detection, membranes were incubated with HRP-conjugated anti-rabbit antibody or anti-goat antibody at room temperature for 1 h and washed 3 times with TBST buffer. The blots were soaked in ECL Plus reagent for 5 min and exposed to KODAK x-ray film. Signals were identified and quantified using a Typhoon Scanner and Densitometer from Amersham Biosciences (Piscataway, N.J.). Data was analyzed by ANOVA using the Newman-Keuls test to assess significance.
Results
cGMP Production in Cardiac Fibroblasts

To determine if NPRA was expressed in the cultured fibroblast cells, cGMP accumulation assays were utilized. BNP dose-dependently induced intracellular cyclic GMP production in cardiac fibroblasts with an EC50 of 50 nmol/L. These results are consistent with the report of Cao and Gardner showing NPRA expression in cardiac fibroblasts.

Effects of BNP on TGFβ-Induced Fibroblast Proliferation

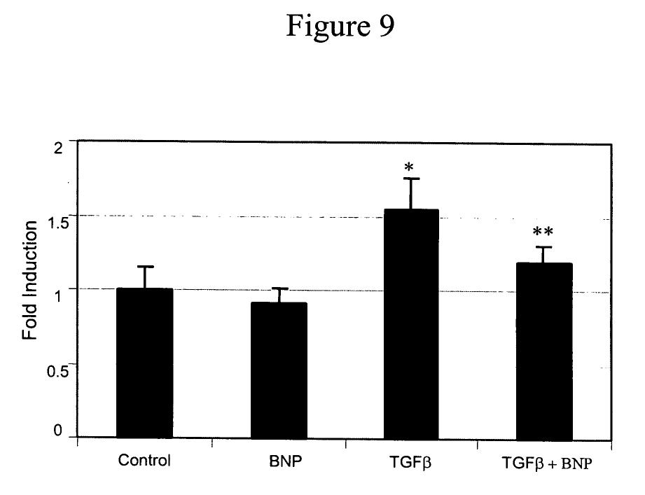

To examine the effects of TGFβ and BNP on cell proliferation, BrdU incorporation was measured in cardiac fibroblasts treated with TGFβ in the presence or absence of BNP. TGFβ modestly increased (˜50%) cardiac fibroblast proliferation, and BNP inhibited TGFβ-induced proliferation by ˜65% (FIG. 9).

Effects of BNP on TGFβ-Induced Gene Expression

In order to determine the effects of BNP on gene expression profiles induced by TGFT in cardiac fibroblasts, a microarray analysis was performed. Fluorescently-labeled cDNA probes were prepared from pooled mRNAs generated from duplicate wells of cells from four groups: unstimulated (control), TGFβ-treated, BNP-treated, and co-treated with TGFβ and BNP for 24 and 48 h (as described above). Arrays were probed in duplicate for a total of 12 hybridizations (6 at each time point): control compared to TGFβ-treated, TGFβ-treated compared to TGFβ+BNP-treated, and control compared to BNP-treated.

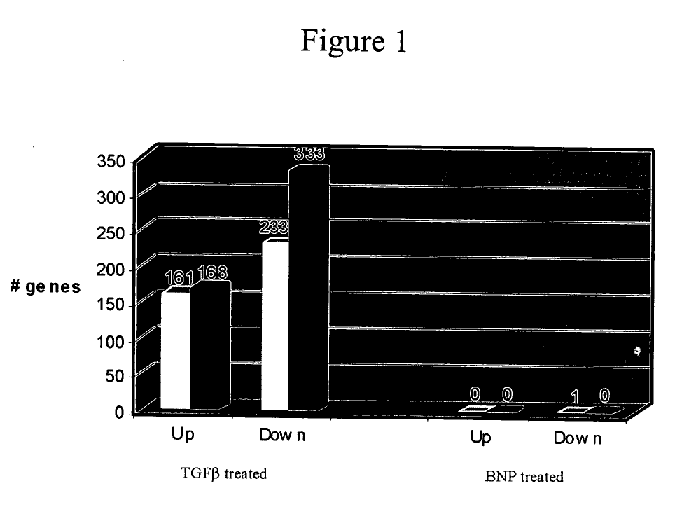

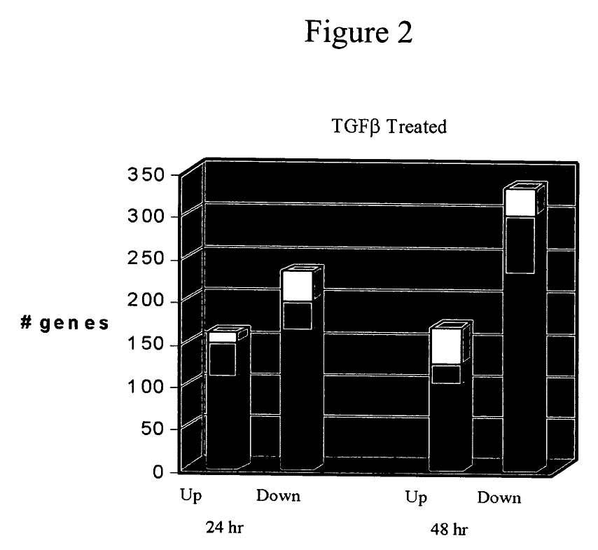

It was observed that BNP had no significant effects on gene expression in unstimulated human cardiac fibroblasts (FIG. 1). In contrast, TGFβ induced 394 and 501 gene expression changes at 24 and 48 h, respectively. These differentially expressed genes represent ˜7-8% of the target genes on the array. Interestingly, BNP had dramatic effects on the gene expression changes induced by TGFβ (FIG. 2). Approximately, 88% and 85% of TGFβ-regulated gene expression events were opposed by BNP at 24 and 48 h, respectively. These results demonstrate that BNP has strikingly different effects on gene expression in TGFβ stimulated fibroblasts compared to unstimulated cells.

Gene Expression Clustering

To identify different gene expression patterns following TGFβ stimulation, we performed a hierarchical cluster analysis. A visualization of this analysis is shown in FIG. 3. A complete listing of differentially expressed genes is provided in Table 2. The clustered expression patterns showed temporal effects of TGFβ responsive genes (compare A to B). In addition, the dramatic effects of BNP in opposing TGFβ induced up- and down-regulated gene changes were revealed in the clusters (compare A and B to C and D). The insignificant effects of BNP on gene expression in unstimulated cardiac fibroblast cells were evident in groups E and F.

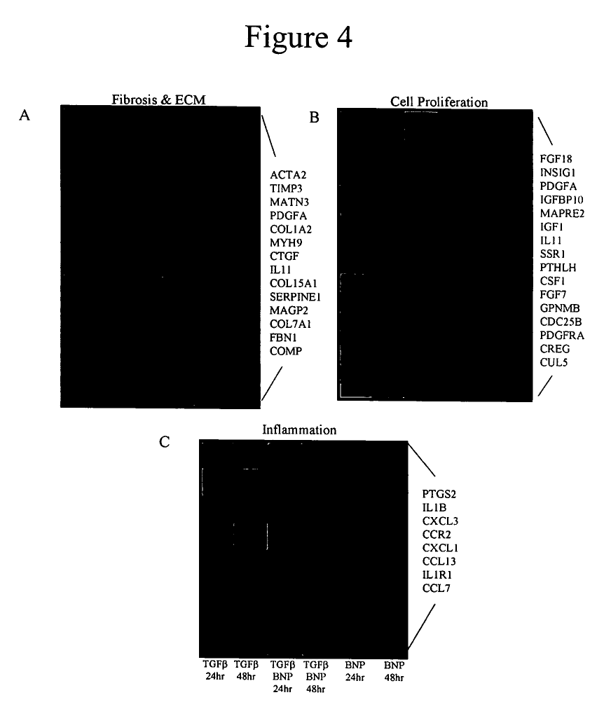

Genes were grouped according to functional categories by using a combination of gene expression clustering and functional annotations. A cluster of genes involved in fibrosis and ECM production was up-regulated in cells stimulated with TGFβ; these genes were down-regulated when treated with BNP (FIG. 4a). This cluster includes extracellular matrix components: Collagen 1a2 (COL1A2), Collagen 15A (COL15A), Collagen 7A1 (COL7A1), Microfibril-associated glycoprotein-2 (MAGP2), Matrilin 3 (MATN3), Fibrillin 1 (FBN1), and Cartilage oligomeric matrix protein (COMP). Also included in the cluster are known markers of fibrosis such as TIMP3, CTGF, IL11, and SERPINE1 (PAI-1). Furthermore, the cluster revealed that BNP opposed TGFβ-induction of myofibroblast markers including α-smooth muscle actin 2 (ACTA2) and non-muscle myosin heavy chain (MYH9).

Many genes involved in cell proliferation were also regulated by TGFβ and were opposed by BNP (FIG. 4B). For example, TGFβ induced the expression of positive regulators of cell proliferation, including PDGFA, IGFBP10, IGF1, and Parathyroid hormone-like hormone (PTHLH). It was also found that TGFβ down-regulated both positive and negative regulators of proliferation, such as, CDC25B and Cullin 5 (CUL5), respectively. All of these TGFβ-regulated gene events were opposed by BNP.

BNP affected TGFβ-induced genes involved in inflammation (FIG. 4C). For example, BNP reversed TGFβ-induction of PTGS2 (COX2), TNF α-induced protein 6 (TNFAIP6), and TNF superfamily, member 4 (TNFSF4) (FIG. 4C and data not shown). TNFAIP6 and TNFSF4 were not included in FIG. 4C, since some of the data points at 48 h did not meet acceptable criteria (see Experimental); at 24 h both genes were elevated ˜3-fold by TGFβ and opposed by BNP. TGFβ also down-regulated many pro-inflammatory genes including IL1B, CCR2 (MCP1-R), CXCL1 (GRO1), CXCL3 (GRO3), and CCL13 (MCP4), which were reversed by BNP. The significance of these inflammatory changes is discussed below.

TABLE 2
Expression data for differentially expressed genes in TGFβ-treated human cardiac fibroblasts. Median
differential expression values are shown for each hybridization: control vs. TGFβ 24 h (column 2); control vs.
TGFβ 48 h (column 3); TGFβ vs. TGFβ + BNP 24 h (column 4); TGFβ vs.
TGFβ + BNP 48 h (column 5); control vs. BNP 24 h (column 6); and control vs. BNP 48 h (column 7).

| | | TGF | | | TGF | | | | |
|---|---|---|---|---|---|---|---|---|---|
| | TGF | BNP | BNP | TGF | BNP | BNP | | | |
| Clone ID | 24 h | 24 h | 24 h | 48 h | 48 h | 48 h | Symbol | Name | Accession |
| P00777_A03 | 2.5 | −2.8 | 1.1 | 1.5 | −1.6 | 1.1 | | EST | |
| P00777_A04 | 8.9 | −5.7 | 1.2 | 3.3 | −2.4 | 1 | | EST | |
| P00777_A12 | 2.1 | −2.4 | −1 | 1.8 | −1.9 | −1.1 | | EST | |
| P01061_E01 | 2.7 | −3 | 1 | 2.6 | −2.8 | −1 | | EST | |
| P01061_B10 | −2.7 | 2.3 | 1.1 | −4 | 2.4 | −1.2 | | EST | |
| P01077_A08 | −1.8 | 3.1 | 1.3 | −2.2 | 1.9 | 1.2 | | No Sequence | |
| P01111_A08 | −1.3 | 1.4 | 1.3 | −1.8 | 1.7 | 1.1 | | EST | |
| P01113_E11 | −1.7 | 1.8 | 1.1 | −1.8 | 1.6 | −1 | | EST | |
| P01111_F07 | −4.5 | 5.5 | 1.3 | −5.3 | 4.2 | 1.1 | | EST | |
| P01111_A07 | 2 | −2.7 | 1.3 | 1.4 | −1.5 | −1.1 | | EST | |
| P01110_G03 | −1.2 | 1.5 | 1.3 | −3.9 | 2.1 | 1.1 | | No Sequence | |
| P01108_G07 | 4.2 | −4.4 | −1.1 | 3.9 | −4.5 | −1 | | EST | |
| P01099_G03 | −1.9 | 1.9 | 1.1 | −2.2 | 1.9 | 1.2 | | EST | |
| P01113_B03 | 6.4 | −5.1 | 1 | 4.3 | −3.7 | −1 | | EST | |
| P01080_A11 | 4 | −3 | 1 | 4.2 | −4.1 | −1 | | EST | |
| P01076_E01 | −1.7 | 1.8 | 1.1 | −1.8 | 1.8 | −1.1 | | EST | |
| P01075_H09 | −3.1 | 3.6 | 1.4 | −2.9 | 3.2 | 1.4 | | No Sequence | |
| P01139_D10 | 3 | −2.6 | 1.1 | 2.1 | −2.1 | 1 | | EST | |
| P01132_B01 | −2.1 | 2 | 1 | −1.4 | 1.3 | 1 | | EST | |
| P01123_H03 | 2.2 | −2.2 | 1.2 | 1.9 | −1.9 | 1.1 | | EST | |
| P01117_D08 | −1.7 | 1.5 | 1.1 | −4.9 | 2.4 | −1 | | EST | |
| P01115_F08 | −2.2 | 1.6 | −1 | −2.3 | 1.7 | −1 | | EST | |
| P01081_F02 | 2.4 | −1.8 | 1.2 | 2.4 | −2.1 | 1.1 | | No Sequence | |
| P01087_A12 | 2.4 | −2 | 1 | 2.6 | −2.6 | −1 | | EST | |
| P01077_A02 | 2.2 | −2 | 1 | 1.4 | −1.3 | −1 | | No Sequence | |
| P01136_G11 | −2 | 2.5 | 1.3 | −3 | 2.5 | 1 | | EST | |
| P01130_B03 | −3.3 | 3.5 | 1.1 | −4.2 | 5.3 | 1.1 | | EST | |
| P01124_A05 | −1.2 | −1 | 1.1 | −1.8 | 1.5 | 1 | | EST | |
| P01124_A10 | 2.1 | −2 | −1 | 2.7 | −2.5 | −1.1 | | EST | |
| P01124_B04 | −1.9 | 2 | 1.3 | −1.6 | 1.7 | 1.1 | | EST | |
| P01120_G06 | −2.3 | 2.2 | −1.1 | −2.4 | 2.2 | −1.1 | | EST | |
| P01117_B11 | 1.8 | −2.4 | 1 | 2.4 | −2 | 1 | | EST | |
| P01116_A02 | −3.1 | 2.7 | 1.1 | −3.7 | 2.2 | −1.4 | | EST | |
| P01088_C10 | 2.1 | −2 | −1 | 1.6 | −2.1 | −1.1 | | EST | |
| P01093_C04 | 2.6 | −2.3 | 1 | 1.8 | −1.9 | −1 | | EST | |
| P01095_H01 | −1.8 | 1.8 | 1 | −1.4 | 1.2 | 1 | | EST | |
| P01099_D03 | 1.9 | −1.8 | 1.1 | 1.1 | −1.2 | 1.1 | | EST | |
| P01100_A07 | 1 | −1 | 1.1 | −3 | 1.7 | −1.1 | | EST | |
| P01100_D09 | −1.6 | 1.6 | −1 | −2.1 | 1.8 | −1.1 | | EST | |
| P01101_C11 | −2.4 | 1.7 | −1 | −1.4 | 1.6 | 1 | | No Sequence | |
| P01101_E11 | −1.4 | 1.5 | 1.1 | −2 | 1.8 | −1 | | EST | |
| P01103_H04 | −3.2 | 2.9 | 1.1 | −5.6 | 4.3 | −1 | | EST | |
| P01104_A09 | −1.9 | 1.6 | 1.1 | −1.8 | 1.5 | 1 | | No Sequence | |
| P01104_E03 | −2.5 | 2.3 | −1 | −2.8 | 2 | −1.1 | | EST | |
| P01104_G04 | 2.5 | −2 | −1 | 1.1 | −1.3 | −1.1 | | EST | |
| P01104_G12 | −3.7 | 2.7 | −1.1 | −4.9 | 3.2 | −1 | | EST | |
| P01105_A05 | 2.3 | −2.3 | 1.3 | 1.3 | −1.3 | 1 | | EST | |
| P01105_D09 | 1.8 | −1.1 | 1.1 | 1.8 | −2.1 | −1 | | EST | |
| P01109_A01 | −1.4 | 1.4 | 1.2 | −2.2 | 1.7 | 1.1 | A2M | alpha-2-macroglobulin | NM_000014 |
| P01109_G11 | 1.4 | −1 | 1.1 | 2 | −1.6 | 1 | ABCG1 | ATP-binding cassette, sub- | NM_004915 |
| | | | | | | | | family G (WHITE), member 1 | |
| P01092_E08 | 2.3 | −2 | 1.2 | 1.5 | −1.3 | 1.1 | ACLY | ATP citrate lyase | NM_001096 |
| P01088_C02 | −1.9 | 1.8 | 1.2 | −2.1 | 2 | 1 | ACO1 | aconitase 1, soluble | NM_002197 |
| P00777_G09 | 2.6 | −2.2 | −1.5 | 1 | 1 | −1.3 | ACTA1 | actin, alpha 1, skeletal muscle | NM_001100 |
| P01094_F04 | 2.6 | −2.5 | −1.4 | −1 | −1 | −1.4 | ACTA2 | actin, alpha 2, smooth muscle, | NM_001613 |
| | | | | | | | | aorta | |
| P01091_G04 | 1.9 | −1.6 | 1.1 | 1.2 | −1.3 | −1 | ACTR3 | ARP3 actin-related protein 3 | NM_005721 |
| | | | | | | | | homolog (yeast) | |
| P01096_D02 | −1.3 | 1.5 | 1.1 | −2.3 | 2.2 | 1 | ADAMTS1 | a disintegrin-like and | NM_006988 |
| | | | | | | | | metalloprotease (reprolysin | |
| | | | | | | | | type) with thrombospondin | |
| | | | | | | | | type 1 motif, 1 | |
| P01097_D04 | 1.7 | −1.9 | −1 | 2.1 | −1.8 | −1.1 | ADAMTS6 | a disintegrin-like and | NM_014273 |
| | | | | | | | | metalloprotease (reprolysin | |
| | | | | | | | | type) with thrombospondin | |
| | | | | | | | | type 1 motif, 6 | |
| P01092_D03 | −6.5 | 6 | −1.1 | −6.3 | 6.5 | −1 | ADFP | adipose differentiation-related | NM_001122 |
| | | | | | | | | protein | |
| P01070_D09 | −5 | 4.1 | 1.3 | −9.7 | 3.8 | 1.3 | ADH1B | alcohol dehydrogenase IB | NM_000668 |
| | | | | | | | | (class I), beta polypeptide | |
| P01134_D11 | −1.7 | 2 | 1.3 | −3.6 | 1.6 | 1.2 | ADH1C | alcohol dehydrogenase 1C | NM_000669 |
| | | | | | | | | (class I), gamma polypeptide | |
| P01070_D05 | −1.3 | −1.4 | 1.2 | −2.2 | 1.7 | 1.1 | ADH5 | alcohol dehydrogenase 5 | NM_000671 |
| | | | | | | | | (class III), chi polypeptide | |
| P01094_D10 | −2.3 | 2.5 | 1.1 | −2.2 | 1.8 | −1 | ADORA2B | adenosine A2b receptor | NM_000676 |
| P01124_F09 | −1.5 | 1.6 | 1.1 | −1.8 | 1.9 | 1 | AHR | aryl hydrocarbon receptor | NM_001621 |
| P01101_B03 | −2.4 | 1 | 1 | −3 | 2.8 | 1.1 | AKAP2 | A kinase (PRKA) anchor | NM_007203 |
| | | | | | | | | protein 2 | |
| P01120_C03 | −1.9 | 2 | 1.2 | −1.2 | 1.5 | 1.2 | AKR1B1 | aldo-keto reductase family 1, | NM_001628 |
| | | | | | | | | member B1 (aldose reductase) | |
| P01134_B08 | −2.7 | 2.6 | 1.1 | −1.4 | 1.9 | 1.2 | AKR1B10 | aldo-keto reductase family 1, | NM_020299 |
| | | | | | | | | member B10 (aldose | |
| | | | | | | | | reductase) | |
| P01069_C01 | −2.8 | 3.1 | 1.2 | −2.2 | 2.6 | 1.1 | AKR1C1 | aldo-keto reductase family 1, | NM_001353 |
| | | | | | | | | member C1 (dihydrodiol | |
| | | | | | | | | dehydrogenase 1; 20-alpha (3- | |
| | | | | | | | | alpha)-hydroxysteroid | |
| | | | | | | | | dehydrogenase) | |
| P01081_A11 | −2.3 | 3.3 | 1.6 | −2.2 | 1.9 | 1.3 | AKR1C2 | aldo-keto reductase family 1, | NM_001354 |
| | | | | | | | | member C2 (dihydrodiol | |
| | | | | | | | | dehydrogenase 2; bile acid | |
| | | | | | | | | binding protein; 3-alpha | |
| | | | | | | | | hydroxysteroid | |
| | | | | | | | | dehydrogenase, type III) | |
| P01143_D10 | −2.8 | 3.2 | 1.3 | −2.1 | 2.7 | 1.1 | AKR1C2 | aldo-keto reductase family 1, | NM_001354 |
| | | | | | | | | member C2 (dihydrodiol | |
| | | | | | | | | dehydrogenase 2; bile acid | |
| | | | | | | | | binding protein; 3-alpha | |
| | | | | | | | | hydroxysteroid | |
| | | | | | | | | dehydrogenase, type III) | |
| P01106_C11 | −2.3 | 2.8 | 1.2 | −2 | 2.5 | 1.1 | AKR1C3 | aldo-keto reductase family 1, | NM_003739 |
| | | | | | | | | member C3 (3-alpha | |
| | | | | | | | | hydroxysteroid | |
| | | | | | | | | dehydrogenase, type II) | |
| P01094_D12 | −2.8 | 3.6 | 1.2 | −2.5 | 1.7 | 1.2 | ALDH1A3 | aldehyde dehydrogenase 1 | NM_000693 |
| | | | | | | | | family, member A3 | |
| P01094_E01 | −1.4 | 1.8 | 1.1 | −2.1 | 1.6 | 1.1 | ALDH3A2 | aldehyde dehydrogenase 3 | NM_000382 |
| | | | | | | | | family, member A2 | |
| P01140_G11 | −1.9 | 2.7 | 1.4 | −2.5 | 1.8 | 1.1 | ALDH3A2 | aldehyde dehydrogenase 3 | NM_000382 |
| | | | | | | | | family, member A2 | |
| P01118_A12 | −1.9 | 1.6 | 1.1 | −2.6 | 2.2 | 1 | ALEX1 | ALEX1 protein | NM_016608 |
| P01096_E12 | −2.4 | 2 | 1 | −2.1 | 2.2 | 1 | ANG | angiogenin, ribonuclease, | NM_001145 |
| | | | | | | | | RNase A family, 5 | |
| P01145_E08 | −2 | 2.3 | 1.2 | −2.9 | 2.6 | −1 | ANGPT1 | angiopoietin 1 | NM_001146 |
| P01091_G02 | −1.2 | 1.5 | 1.2 | −2.7 | 2 | 1.1 | ANGPT2 | angiopoietin 2 | NM_001147 |
| P01094_D06 | −2.1 | 1.9 | −1 | −1.9 | 1.3 | −1.1 | ANK3 | ankyrin 3, node of Ranvier | NM_001149 |
| | | | | | | | | (ankyrin G) | |
| P01128_A07 | −1.5 | 1.8 | 1.2 | −2.2 | 2.3 | 1.2 | AOX1 | aldehyde oxidase 1 | NM_001159 |
| P01116_H05 | −1.1 | 1.4 | 1.2 | −2 | 1.8 | 1 | APELIN | apelin; peptide ligand for APJ | NM_017413 |
| | | | | | | | | receptor | |
| P01103_F06 | 2.4 | −2.4 | −1.1 | 1.4 | −1.5 | −1.1 | APG3 | autophagy Apg3p/Aut1p-like | NM_022488 |
| P01123_A07 | 3.2 | −3 | −1 | 1.5 | −1.8 | −1 | APOA1 | apolipoprotein A-I | NM_000039 |
| P01105_G06 | −2.2 | 1.8 | −1.1 | −4.5 | 5.7 | 1.1 | APOC1 | apolipoprotein C-I | NM_001645 |
| P01124_G03 | −1.3 | 1.4 | 1 | −2.4 | 2 | 1.1 | APOE | apolipoprotein E | NM_000041 |
| P01105_B02 | −1.6 | 1.8 | −1 | −2.9 | 1.9 | 1.2 | ARHGAP6 | Rho GTPase activating protein 6 | NM_001174 |
| P01064_G03 | −1.1 | 1.3 | 1.1 | −2 | 1.6 | 1.2 | ARHGEF16 | Rho guanine exchange factor | NM_014448 |
| | | | | | | | | (GEF) 16 | |
| P01110_E10 | −2 | 2.1 | 1.2 | −2.3 | 1.9 | 1 | ARHGEF3 | Rho guanine nucleotide | NM_019555 |
| | | | | | | | | exchange factor (GEF) 3 | |
| P01142_C03 | −1.6 | 1.8 | 1.5 | −1.9 | 1.7 | 1.2 | ARHI | ras homolog gene family, | NM_004675 |
| | | | | | | | | member I | |
| P01138_A09 | 1.9 | −2.2 | −1.1 | 1.8 | −1.9 | −1.1 | ARL4 | ADP-ribosylation factor-like 4 | NM_005738 |
| P01064_G12 | −1.7 | 1.8 | 1.1 | −1.8 | 1.6 | −1 | ARNT2 | aryl-hydrocarbon receptor | NM_014862 |
| | | | | | | | | nuclear translocator 2 | |
| P01088_H09 | −1.5 | 1.7 | 1.2 | −1.8 | 1.6 | 1.1 | ASAH1 | N-acylsphingosine | NM_004315 |
| | | | | | | | | amidohydrolase (acid | |
| | | | | | | | | ceramidase) 1 | |
| P01105_F06 | 2.9 | −2.8 | 1.1 | 2.1 | −2.4 | −1.2 | ASNS | asparagine synthetase | NM_001673 |
| P01070_E06 | 1.8 | −1.5 | −1.3 | 1.6 | −1.4 | 1 | ATF3 | activating transcription factor 3 | NM_001674 |
| P01122_G07 | −1.2 | 1.7 | 1.2 | −1.8 | 1.5 | 1.3 | AXIN2 | axin 2 (conductin, axil) | NM_004655 |
| P01115_D06 | −1.4 | 1.6 | 1 | −2 | 1.5 | −1.1 | B3GALT2 | UDP-Gal:betaGlcNAc beta | NM_003783 |
| | | | | | | | | 1,3-galactosyltransferase, | |
| | | | | | | | | polypeptide 2 | |
| P01128_A08 | −1.6 | 1.7 | 1 | −2.4 | 1.7 | −1 | B3GALT3 | UDP-Gal:betaGlcNAc beta | NM_003781 |
| | | | | | | | | 1,3-galactosyltransferase, | |
| | | | | | | | | polypeptide 3 | |
| P01095_F06 | 2.4 | −2.2 | 1.1 | 1.3 | −1.5 | −1 | BAI3 | brain-specific angiogenesis | NM_001704 |
| | | | | | | | | inhibitor 3 | |
| P01094_C02 | −1.8 | 2 | 1.2 | −2.4 | 2.7 | −1 | BF | B-factor, properdin | NM_001710 |
| P01134_E02 | −1.7 | 1.8 | 1 | −2.2 | 1.6 | −1 | BFSP1 | beaded filament structural | NM_001195 |
| | | | | | | | | protein 1, filensin | |
| P01081_D08 | −1.2 | 1.7 | 1.2 | −3.5 | 1.8 | 1.2 | BIRC1 | baculoviral IAP repeat- | NM_004536 |
| | | | | | | | | containing 1 | |
| P01094_B06 | −2.6 | 2.9 | 1.1 | −4 | 2.5 | −1 | BMP4 | bone morphogenetic protein 4 | NM_001202 |
| P01145_A02 | −3.2 | 2.3 | 1 | −3.6 | 3.2 | −1.1 | BNIP2 | BCL2/adenovirus E1B 19 kDa | NM_004330 |
| | | | | | | | | interacting protein 2 | |
| P01075_F05 | −1.5 | 1.5 | 1.2 | −1.8 | 2 | 1.2 | BRE | brain and reproductive organ- | NM_004899 |
| | | | | | | | | expressed (TNFRSF1A | |
| | | | | | | | | modulator) | |
| P01124_B10 | −1.3 | 1.5 | 1.3 | −2.2 | 1.6 | 1.2 | BST1 | bone marrow stromal cell | NM_004334 |
| | | | | | | | | antigen 1 | |
| P01094_B08 | −1.8 | 1.6 | −1.1 | −1.2 | 1.3 | −1.1 | BTD | biotinidase | NM_000060 |
| P01093_E08 | −2 | 1.5 | −1.1 | −1.9 | 2.7 | 1.1 | C1R | complement component 1, r | NM_001733 |
| | | | | | | | | subcomponent | |
| P01077_E12 | −1.4 | 1.6 | 1.1 | −1.8 | 1.9 | −1.1 | C1S | complement component 1, s | NM_001734 |
| | | | | | | | | subcomponent | |
| P01097_G03 | 1.9 | −1.7 | −1 | 1 | −1.5 | −1.1 | C20orf14 | chromosome 20 open reading | NM_012469 |
| | | | | | | | | frame 14 | |
| P01140_A07 | 2.3 | −3.2 | −1 | 3 | −2.6 | −1 | C20orf97 | chromosome 20 open reading | NM_021158 |
| | | | | | | | | frame 97 | |
| P01069_E02 | −1.7 | 1.6 | 1.1 | −3.3 | 3.2 | 1.1 | C6 | complement component 6 | NM_000065 |
| P01077_E10 | −3.1 | 2.9 | 1.1 | −8.2 | 4.7 | −1 | C7 | complement component 7 | NM_000587 |
| P01099_C10 | −1.8 | 2.1 | 1.2 | −2.7 | 3.5 | 1.1 | CA12 | carbonic anhydrase XII | NM_001218 |
| P01117_G05 | −3 | 2.4 | −1.1 | −2.2 | 2.3 | 1.1 | CAMK2B | calcium/calmodulin-dependent | NM_001220 |
| | | | | | | | | protein kinase (CaM kinase) II | |
| | | | | | | | | beta | |
| P01114_A05 | −2.7 | 3.9 | 1.2 | −3.5 | 2.7 | 1 | CAMK2D | calcium/calmodulin-dependent | NM_001221 |
| | | | | | | | | protein kinase (CaM kinase) II | |
| | | | | | | | | delta | |
| P01080_B05 | −2.3 | 3 | 1.1 | −2.3 | 2.1 | 1.1 | CAMK2D | calcium/calmodulin-dependent | NM_001221 |
| | | | | | | | | protein kinase (CaM kinase) II | |
| | | | | | | | | delta | |
| P01063_E07 | −1.6 | 2 | 1.2 | −1.8 | 1.6 | 1.1 | CASP1 | caspase 1, apoptosis-related | NM_001223 |
| | | | | | | | | cysteine protease (interleukin | |
| | | | | | | | | 1, beta, convertase) | |
| P01093_G08 | −2.4 | 2.3 | −1.2 | −2.1 | 2.4 | 1 | CAV1 | caveolin 1, caveolae protein | NM_001753 |
| | | | | | | | | 22 kDa | |
| P01093_E04 | 1.8 | −1.7 | −1.1 | 1.6 | −1.9 | −1.1 | CBS | cystathionine-beta-synthase | NM_000071 |
| P01064_D02 | −1.5 | 1.6 | −1.3 | −2.2 | 2.8 | −1.1 | CCL13 | chemokine (C—C motif) ligand | NM_005408 |
| | | | | | | | | 13 | |
| P01072_E08 | −1.3 | 1.4 | −1.2 | −2.2 | 3.2 | −1.1 | CCL7 | chemokine (C—C motif) ligand 7 | NM_006273 |
| P01127_H03 | 1.1 | 1.2 | −1.3 | −2 | 2.9 | −1 | CCL8 | chemokine (C—C motif) ligand 8 | NM_005623 |
| P01070_A04 | −1.4 | 1.9 | 1.2 | −3.2 | 2.4 | 1.1 | CCR2 | chemokine (C—C motif) | NM_000647 |
| | | | | | | | | receptor 2 | |
| P01138_B02 | −1.2 | 1.3 | 1.3 | −3.6 | 1.5 | 1 | CCRL1 | chemokine (C—C motif) | NM_016557 |
| | | | | | | | | receptor-like 1 | |
| P01069_H09 | −1.9 | 1.9 | 1.3 | −3.6 | 1.8 | 1.2 | CD36 | CD36 antigen (collagen type I | NM_000072 |
| | | | | | | | | receptor, thrombospondin | |
| | | | | | | | | receptor) | |
| P01072_E03 | −2.8 | 2.7 | 1.2 | −2.9 | 2.8 | 1.2 | CDC25B | cell division cycle 25B | NM_004358 |
| P01093_H07 | 2 | −4.3 | 1.2 | 2.1 | −2 | −1 | CDH2 | cadherin 2, type 1, N-cadherin | NM_001792 |
| | | | | | | | | (neuronal) | |
| P01129_E07 | 1.7 | −1.4 | 1.1 | 2 | −1.9 | −1.1 | CDH4 | cadherin 4, type 1, R-cadherin | NM_001794 |
| | | | | | | | | (retinal) | |
| P01130_H07 | 2.1 | −2.4 | −1.1 | 1.9 | −1.8 | −1 | CDH5 | cadherin 5, type 2, VE- | NM_001795 |
| | | | | | | | | cadherin (vascular epithelium) | |
| P01116_H02 | −3.3 | 2.1 | 1.1 | −2 | 2.4 | 1.1 | CDK5RAP2 | CDK5 regulatory subunit | NM_018249 |
| | | | | | | | | associated protein 2 | |
| P01102_B02 | −2.1 | 2.5 | 1 | −3.4 | 3.2 | −1.1 | CDSN | comeodesmosin | NM_001264 |
| P01140_G02 | −1.4 | 1.3 | 1.1 | −2.9 | 2.4 | 1 | CEACAM5 | carcinoembryonic antigen- | NM_004363 |
| | | | | | | | | related cell adhesion molecule 5 | |
| P01094_A06 | −1.6 | 1.3 | 1.3 | −4.2 | 2.9 | 1 | CEACAM5 | carcinoembryonic antigen- | NM_004363 |
| | | | | | | | | related cell adhesion molecule 5 | |
| P01062_G02 | −1.3 | 1.5 | 1.3 | −2.9 | 2.1 | 1.1 | CEACAM6 | carcinoembryonic antigen- | NM_002483 |
| | | | | | | | | related cell adhesion molecule | |
| | | | | | | | | 6 (non-specific cross reacting | |
| | | | | | | | | antigen) | |
| P01099_B05 | −1.8 | 1.8 | 1.1 | −2.9 | 3 | −1.1 | CEACAM7 | carcinoembryonic antigen- | NM_006890 |
| | | | | | | | | related cell adhesion molecule 7 | |
| P01090_E04 | −1.3 | 1.6 | 1.3 | −1.9 | 1.8 | −1 | CEBPD | CCAAT/enhancer binding | NM_005195 |
| | | | | | | | | protein (C/EBP), delta | |
| P01070_A01 | −2.6 | 3.1 | −1 | −9.2 | 9.2 | 1.1 | CHI3L1 | chitinase 3-like 1 (cartilage | NM_001276 |
| | | | | | | | | glycoprotein-39) | |
| P01125_G02 | −2.9 | 2 | 1 | −5 | 6.2 | 1 | CHI3L2 | chitinase 3-like 2 | NM_004000 |
| P01134_F10 | 8 | −6.3 | 1.2 | 19.5 | −8 | 1.1 | CILP | cartilage intermediate layer | NM_003613 |
| | | | | | | | | protein, nucleotide | |
| | | | | | | | | pyrophosphohydrolase | |
| P01089_A12 | −1.9 | 2.1 | 1 | −2.1 | 2.1 | −1 | CITED2 | Cbp/p300-interacting | NM_006079 |
| | | | | | | | | transactivator, with Glu/Asp- | |
| | | | | | | | | rich carboxy-terminal domain, 2 | |
| P01076_A07 | 2.1 | −1.8 | −1 | 1.4 | −1.2 | 1.1 | CKAP4 | cytoskeleton-associated | NM_006825 |
| | | | | | | | | protein 4 | |
| P01104_C09 | 2.2 | −2.4 | 1.1 | 4.3 | −2.8 | 1 | CKLF | chemokine-like factor | NM_016326 |
| P01103_G05 | −1.4 | 1.6 | 1.3 | −2.5 | 1.5 | 1.3 | CLDN1 | claudin 1 | NM_021101 |
| P01105_D03 | −3 | 2.7 | 1.3 | −2.6 | 2 | −1 | CLECSF2 | C-type (calcium dependent, | NM_005127 |
| | | | | | | | | carbohydrate-recognition | |
| | | | | | | | | domain) lectin, superfamily | |
| | | | | | | | | member 2 (activation-induced) | |
| P01064_F09 | 2.2 | −1.5 | 1.2 | 1.2 | −1.2 | 1.1 | CNN1 | calponin 1, basic, smooth | NM_001299 |
| | | | | | | | | muscle | |
| P01090_A03 | −1.1 | 1.3 | 1.2 | −2.2 | 1.6 | 1.1 | CNTNAP1 | contactin associated protein 1 | NM_003632 |
| P01069_F02 | 1.2 | 1.2 | 1.2 | 3.2 | −3 | 1 | COL15A1 | collagen, type XV, alpha 1 | NM_001855 |
| P01077_E08 | 1.8 | −1.5 | 1 | 1.9 | −1.9 | −1 | COL1A2 | collagen, type I, alpha 2 | NM_000089 |
| P01093_F03 | 1.7 | −2.3 | 1 | 1.9 | −2.1 | −1 | COL4A2 | collagen, type IV, alpha 2 | NM_001846 |
| P01105_C12 | 1.8 | −1.5 | 1.4 | 3.1 | −2.3 | 1.1 | COL7A1 | collagen, type VII, alpha 1 | NM_000094 |
| | | | | | | | | (epidermolysis bullosa, | |
| | | | | | | | | dystrophic, dominant and | |
| | | | | | | | | recessive) | |
| P01120_G04 | 2.7 | −2.1 | 1.2 | 3.8 | −3.6 | 1.1 | COL8A2 | collagen, type VIII, alpha 2 | M60832 |
| P01084_A12 | −4.9 | 4.7 | 1.1 | −9.9 | 6 | 1 | COLEC12 | collectin sub-family member | NM_030781 |
| | | | | | | | | 12 | |
| P01082_H06 | 1.3 | −1.3 | 1.2 | 3.3 | −2.4 | 1.2 | COMP | cartilage oligomeric matrix | NM_000095 |
| | | | | | | | | protein | |
| | | | | | | | | (pseudoachondroplasia, | |
| | | | | | | | | epiphyseal dysplasia 1, | |
| | | | | | | | | multiple) | |
| P01129_C12 | 1.4 | −1.5 | 1.3 | 2.6 | −1.6 | 1.3 | COMP | cartilage oligomeric matrix | NM_000095 |
| | | | | | | | | protein | |
| | | | | | | | | (pseudoachondroplasia, | |
| | | | | | | | | epiphyseal dysplasia 1, | |
| | | | | | | | | multiple) | |
| P01076_C09 | −2.2 | 2.7 | 1.2 | −2.1 | 1.6 | 1.1 | COPB | coatomer protein complex, | NM_016451 |
| | | | | | | | | subunit beta | |
| P01085_D11 | −4 | 4.2 | 1.1 | −7.7 | 4.1 | 1.1 | CPA4 | carboxypeptidase A4 | NM_016352 |
| P01104_A07 | −1.9 | 2 | 1.2 | −2.5 | 2.2 | 1 | CPD | carboxypeptidase D | NM_001304 |
| P01077_G01 | 1.9 | −1.8 | 1.1 | 1.7 | −1.9 | 1.1 | CRABP2 | cellular retinoic acid binding | NM_001878 |
| | | | | | | | | protein 2 | |
| P01095_E03 | −1.8 | 1.8 | 1.2 | −2.1 | 2 | −1.1 | CREG | cellular repressor of E1A- | NM_003851 |
| | | | | | | | | stimulated genes | |
| P01124_E01 | −2.2 | 2.1 | 1 | −2.5 | 2.2 | −1 | CREM | cAMP responsive element | NM_001881 |
| | | | | | | | | modulator | |
| P01120_B01 | 1.8 | −1.6 | 1.2 | 3.9 | −3.4 | 1.1 | CRLF1 | cytokine receptor-like factor 1 | NM_004750 |
| P01120_D10 | −1.5 | 1.9 | 1.3 | −3.5 | 2.4 | 1.1 | CROT | camitine O- | NM_021151 |
| | | | | | | | | octanoyltransferase | |
| P01124_F10 | −1.2 | 1.3 | 1.2 | −1.8 | 1.7 | 1.1 | CRYAA | crystallin, alpha A | NM_000394 |
| P00777_A08 | −2 | 1.6 | 1.1 | −2.6 | 2.5 | −1.1 | CRYAB | crystallin, alpha B | NM_001885 |
| P01077_E04 | −2.1 | 1.8 | 1.1 | −2.5 | 2.6 | −1.1 | CRYAB | crystallin, alpha B | NM_001885 |
| P01125_B11 | −1.8 | 1.2 | 1.1 | −1.8 | 1.8 | 1 | CSF1 | colony stimulating factor 1 | NM_000757 |
| | | | | | | | | (macrophage) | |
| P01108_G05 | 3.8 | −3 | 1.1 | 2.3 | −2.4 | 1 | CSPG2 | chondroitin sulfate | NM_004385 |
| | | | | | | | | proteoglycan 2 (versican) | |
| P01075_F12 | −1.5 | 1.6 | 1.1 | −2 | 2 | 1.1 | CSRP2 | cysteine and glycine-rich | NM_001321 |
| | | | | | | | | protein 2 | |
| P01145_A03 | −2.1 | 2.4 | 1 | −3.7 | 3.4 | −1.1 | CST4 | cystatin S | NM_001899 |
| P00777_D03 | 2.5 | −2 | 1.1 | 1.1 | −1.4 | −1.2 | CTGF | connective tissue growth factor | NM_001901 |
| P01077_D08 | 2.6 | −3.5 | −1.2 | 1.8 | −2.7 | −1.2 | CTGF | connective tissue growth factor | NM_001901 |
| P01069_D11 | 2 | −2.1 | 1.2 | 1.9 | −1.4 | 1.2 | CTH | cystathionase (cystathionine | NM_001902 |
| | | | | | | | | gamma-lyase) | |
| P01099_B01 | −1.7 | 2 | 1.1 | −2 | 1.6 | −1 | CTNNAL1 | catenin (cadherin-associated | NM_003798 |
| | | | | | | | | protein), alpha-like 1 | |
| P01093_G10 | −1.4 | 1.4 | 1.1 | −1.8 | 2 | 1 | CTSC | cathepsin C | NM_001814 |
| P01077_G03 | −1 | 1.3 | 1.2 | −1.8 | 1.7 | 1.2 | CTSH | cathepsin H | NM_004390 |
| P01069_H12 | −1.5 | 1.5 | 1.1 | −2.3 | 2.6 | −1 | CTSK | cathepsin K (pycnodysostosis) | NM_000396 |
| P01093_G09 | −2.5 | 2.1 | 1.1 | −2 | 2.3 | −1 | CTSL | cathepsin L | NM_001912 |
| P01112_D02 | −1.6 | 1.8 | 1.2 | −2.9 | 2.1 | −1 | CUGBP2 | CUG triplet repeat, RNA | NM_006561 |
| | | | | | | | | binding protein 2 | |
| P01131_G04 | −1.3 | 1.6 | 1.3 | −2.2 | 1.7 | 1.3 | CUGBP2 | CUG triplet repeat, RNA | NM_006561 |
| | | | | | | | | binding protein 2 | |
| P01090_H01 | −2 | 1.8 | 1.3 | −1.5 | 1.9 | 1.3 | CUL5 | cullin 5 | NM_003478 |
| P01085_C05 | −3.8 | 3.4 | 1.1 | −5.5 | 5 | −1 | CXCL1 | chemokine (C—X—C motif) | NM_001511 |
| | | | | | | | | ligand 1 (melanoma growth | |
| | | | | | | | | stimulating activity, alpha) | |
| P01093_A02 | −3.7 | 3.1 | 1 | −5.8 | 5.4 | −1 | CXCL1 | chemokine (C—X—C motif) | NM_001511 |
| | | | | | | | | ligand 1 (melanoma growth | |
| | | | | | | | | stimulating activity, alpha) | |
| P01125_H11 | −2.4 | 2 | 1.1 | −2.3 | 2.1 | 1 | CXCL3 | chemokine (C—X—C motif) | NM_002090 |
| | | | | | | | | ligand 3 | |
| P01136_B01 | −4.5 | 4.4 | 1.1 | −8.4 | 10 | 1.1 | CXCL6 | chemokine (C—X—C motif) | NM_002993 |
| | | | | | | | | ligand 6 (granulocyte | |
| | | | | | | | | chemotactic protein 2) | |
| P01069_D07 | −2.4 | 2.3 | 1.3 | −2 | 1.7 | 1 | CYB5 | cytochrome b-5 | NM_001914 |
| P00777_A11 | 2 | −2.5 | −1 | 1.8 | −2 | −1 | CYR61 | cysteine-rich, angiogenic | NM_001554 |
| | | | | | | | | inducer, 61 | |
| P00777_C11 | 1.8 | −2.5 | −1 | 1.8 | −1.9 | −1.1 | CYR61 | cysteine-rich, angiogenic | NM_001554 |
| | | | | | | | | inducer, 61 | |
| P00777_C12 | 2 | −2.6 | −1.1 | 1.9 | −1.9 | −1.1 | CYR61 | cysteine-rich, angiogenic | NM_001554 |
| | | | | | | | | inducer, 61 | |
| P01108_B04 | 2.3 | −2.4 | 1.1 | 1.9 | −1.9 | −1.1 | CYR61 | cysteine-rich, angiogenic | NM_001554 |
| | | | | | | | | inducer, 61 | |
| P01130_H03 | 2 | −2.4 | −1.1 | 1.9 | −1.8 | −1.1 | CYR61 | cysteine-rich, angiogenic | NM_001554 |
| | | | | | | | | inducer, 61 | |
| P01100_C06 | 2.2 | −2.4 | −1.1 | 1.8 | −1.9 | −1 | DACT1 | dapper homolog 1, antagonist | NM_016651 |
| | | | | | | | | of beta-catenin (xenopus) | |
| P01069_C07 | 1.7 | −1.4 | 1.1 | 2.3 | −2.1 | 1.1 | DAF | decay accelerating factor for | NM_000574 |
| | | | | | | | | complement (CD55, Cromer | |
| | | | | | | | | blood group system) | |
| P01129_B04 | −2.8 | 2.5 | 1.2 | −4.3 | 3.5 | 1 | DAPK1 | death-associated protein | NM_004938 |
| | | | | | | | | kinase 1 | |
| P01092_G02 | −2.7 | 2.6 | 1.1 | −3.8 | 2.8 | 1.2 | DAPK1 | death-associated protein | NM_004938 |
| | | | | | | | | kinase 1 | |
| P01065_A02 | −1.8 | 1.9 | −1 | −1.8 | 1.7 | −1.1 | DDX38 | DEAD/H (Asp-Glu-Ala- | NM_014003 |
| | | | | | | | | Asp/His) box polypeptide 38 | |
| P01105_A10 | −3.7 | 2.9 | −1 | −7 | 5.4 | −1 | DKK1 | dickkopf homolog 1 (Xenopus | NM_012242 |
| | | | | | | | | laevis) | |
| P01113_E05 | 2.8 | −2.4 | −1.1 | 1.8 | −2.1 | −1.1 | DLC1 | deleted in liver cancer 1 | NM_006094 |
| P01093_C11 | −1.8 | 1.9 | −1 | −4.5 | 3.3 | −1 | DPP4 | dipeptidylpeptidase 4 (CD26, | NM_001935 |
| | | | | | | | | adenosine deaminase | |
| | | | | | | | | complexing protein 2) | |
| P01073_G11 | −1.8 | 1.7 | 1 | −1.6 | 1.5 | 1 | DPYSL2 | dihydropyrimidinase-like 2 | NM_001386 |
| P01090_F08 | 1.4 | −1.5 | −1.1 | 2 | −1.9 | −1.1 | DSCR1 | Down syndrome critical region | NM_004414 |
| | | | | | | | | gene 1 | |
| P01122_D11 | 1.7 | −1.2 | 1.3 | 1.9 | −2.1 | 1.3 | EBAF | endometrial bleeding | NM_003240 |
| | | | | | | | | associated factor (left-right | |
| | | | | | | | | determination, factor A; | |
| | | | | | | | | transforming growth factor | |
| | | | | | | | | beta superfamily) | |
| P01123_B11 | −1.8 | 1.7 | 1 | −2.3 | 1.9 | 1 | ECM2 | extracellular matrix protein 2, | NM_001393 |
| | | | | | | | | female organ and adipocyte | |
| | | | | | | | | specific | |
| P01124_E11 | −1.6 | 1.9 | 1.2 | −2.1 | 1.9 | 1.2 | EDG1 | endothelial differentiation, | NM_001400 |
| | | | | | | | | sphingolipid G-protein-coupled | |
| | | | | | | | | receptor, 1 | |
| P01103_G08 | −1.8 | 1.8 | 1.1 | −2.4 | 2.4 | 1 | EDG2 | endothelial differentiation, | NM_001401 |
| | | | | | | | | lysophosphatidic acid G- | |
| | | | | | | | | protein-coupled receptor, 2 | |
| P01093_C01 | −2.1 | 1.5 | −1.1 | −2.9 | 1.9 | −1.3 | EDN1 | endothelin 1 | NM_001955 |
| P01105_H10 | −1.9 | 1.9 | 1 | −2.2 | 2.3 | −1 | EFEMP1 | EGF-containing fibulin-like | NM_004105 |
| | | | | | | | | extracellular matrix protein 1 | |
| P01064_A03 | −1.4 | 1.9 | 1.2 | −2 | 2 | 1.1 | EFNB3 | ephrin-B3 | NM_001406 |
| P01093_B07 | −1.8 | 1.7 | 1.3 | −1.5 | 1.3 | 1.1 | EGR2 | early growth response 2 (Krox- | NM_000399 |
| | | | | | | | | 20 homolog, Drosophila) | |
| P01121_C03 | −2 | 2 | 1.2 | −1.2 | 1.5 | 1.2 | EHD3 | EH-domain containing 3 | NM_014600 |
| P01065_E02 | 1.9 | −1.6 | 1.2 | 3.4 | −3.3 | 1.1 | ELN | elastin (supravalvular aortic | NM_000501 |
| | | | | | | | | stenosis, Williams-Beuren | |
| | | | | | | | | syndrome) | |
| P01096_H11 | −3.4 | 3.7 | 1.1 | −3.5 | 3.2 | −1 | EPAS1 | endothelial PAS domain | NM_001430 |
| | | | | | | | | protein 1 | |
| P01102_E11 | −2 | 2.1 | 1.2 | −2.5 | 2.1 | −1 | EPB41L2 | erythrocyte membrane protein | NM_001431 |
| | | | | | | | | band 4.1-like 2 | |
| P01104_A05 | −2.3 | 3.3 | 1.1 | −2.2 | 2.3 | 1.1 | EPI64 | EBP50-PDZ interactor of 64 kD | NM_031937 |
| P01130_H01 | −2 | 2 | 1.1 | −2.5 | 3.9 | −1 | EPOR | erythropoietin receptor | NM_000121 |
| P01077_A07 | −1.6 | 2.7 | 1.4 | −2.3 | 2.1 | 1.2 | ETV5 | ets variant gene 5 (ets-related | NM_004454 |
| | | | | | | | | molecule) | |
| P01097_C06 | −5.9 | 4.9 | −1 | −15.8 | 14 | −1.1 | EVI2B | ecotropic viral integration site | NM_006495 |
| | | | | | | | | 2B | |
| P01077_A01 | 1.8 | −1.8 | 1.1 | 1.3 | −1.4 | 1 | EXT1 | exostoses (multiple) 1 | NM_000127 |
| P01069_F04 | −1.7 | 1.6 | 1.2 | −2.1 | 1.7 | 1.1 | F2R | coagulation factor II (thrombin) | NM_001992 |
| | | | | | | | | receptor | |
| P01128_B02 | 1.8 | −1.9 | 1.1 | −1 | −1.1 | −1 | F3 | coagulation factor III | NM_001993 |
| | | | | | | | | (thromboplastin, tissue factor) | |
| P01132_G03 | 1.9 | −1.7 | 1.2 | 1.8 | −1.6 | 1.1 | FACL3 | fatty-acid-Coenzyme A ligase, | NM_004457 |
| | | | | | | | | long-chain 3 | |
| P01096_A03 | 1.8 | −2 | 1 | 1.8 | −2 | 1 | FACL3 | fatty-acid-Coenzyme A ligase, | NM_004457 |
| | | | | | | | | long-chain 3 | |
| P01083_D07 | 2.2 | −1.6 | 1.2 | 1.4 | −1.3 | 1 | FADS1 | fatty acid desaturase 1 | NM_013402 |
| P01093_B02 | −2 | 1.6 | 1.1 | −3.4 | 3.4 | 1 | FBLN1 | fibulin 1 | NM_001996 |
| P01123_A08 | 3.4 | −3 | 1.2 | 1.6 | −1.9 | −1 | FBLN5 | fibulin 5 | NM_006329 |
| P01068_H09 | 1.4 | −1.4 | 1 | 2.2 | −2 | −1 | FBN1 | fibrillin 1 (Marfan syndrome) | NM_000138 |
| P01084_E10 | 1.9 | −1.7 | 1.3 | −1.1 | −1.1 | 1.1 | FGF18 | fibroblast growth factor 18 | NM_003862 |
| P01093_B03 | −4.2 | 4.9 | 1.2 | −5.9 | 5.6 | 1 | FGF7 | fibroblast growth factor 7 | NM_002009 |
| | | | | | | | | (keratinocyte growth factor) | |
| P01092_C04 | −3.2 | 2.9 | 1.1 | −3.1 | 2.3 | −1 | FGL2 | fibrinogen-like 2 | NM_006682 |
| P01126_F06 | −5 | 5.2 | −1.1 | −6.5 | 4.9 | −1.1 | FMO2 | flavin containing | NM_001460 |
| | | | | | | | | monooxygenase 2 | |
| P01078_G11 | −1.9 | 2.1 | 1.2 | −3.1 | 2.2 | 1.1 | FMO3 | flavin containing | NM_006894 |
| | | | | | | | | monooxygenase 3 | |
| P01088_F09 | 2 | −1.8 | 1.2 | 1.4 | −1.5 | 1.1 | FOXD1 | forkhead box D1 | NM_004472 |
| P01120_B03 | −1.8 | 1.9 | 1.3 | −1.2 | 1.5 | 1.2 | FRA | Fos-related antigen | NM_024816 |
| P01138_B06 | −1.8 | 1.5 | 1 | −1.4 | 1.8 | −1 | FTHL17 | ferritin, heavy polypeptide-like | NM_031894 |
| | | | | | | | | 17 | |
| P01068_G11 | 2.7 | −2.1 | 1.3 | 2.4 | −2.5 | 1.1 | FUT4 | fucosyltransferase 4 (alpha | NM_002033 |
| | | | | | | | | (1,3) fucosyltransferase, | |
| | | | | | | | | myeloid-specific) | |
| P01077_A05 | 1.8 | −1.5 | 1 | 1.5 | −1.2 | 1 | FYN | FYN oncogene related to | NM_002037 |
| | | | | | | | | SRC, FGR, YES | |
| P01124_G01 | −1.9 | 1.9 | 1.1 | −1.4 | 1.2 | 1 | FZD7 | frizzled homolog 7 | NM_003507 |
| | | | | | | | | (Drosophila) | |
| P01083_B09 | 3.2 | −4 | −1 | 4.2 | −3.5 | −1 | GABARAPL2 | GABA(A) receptor-associated | NM_007285 |
| | | | | | | | | protein-like 2 | |
| P01106_B05 | −1.8 | 1.5 | 1 | −1.1 | 2.4 | 1.2 | GALT | galactose-1-phosphate | NM_000155 |
| | | | | | | | | uridylyltransferase | |
| P01092_G07 | 2.5 | −2.3 | −1.2 | 1.8 | −2.2 | −1.1 | GARS | glycyl-tRNA synthetase | NM_002047 |
| P01085_D09 | −2.9 | 4.1 | 1.4 | −5.3 | 2.6 | 1.3 | GAS1 | growth arrest-specific 1 | NM_002048 |
| P01063_E09 | −2 | 1.7 | 1.1 | −2 | 1.8 | 1.2 | GBP2 | guanylate binding protein 2, | NM_004120 |
| | | | | | | | | interferon-inducible | |
| P01123_D12 | −1.9 | 1.4 | −1.2 | −2.7 | 2.7 | −1.1 | GBP2 | guanylate binding protein 2, | NM_004120 |
| | | | | | | | | interferon-inducible | |
| P01135_C03 | −1.8 | 1.9 | 1.2 | −2.7 | 2.4 | 1.1 | GCNT1 | glucosaminyl (N-acetyl) | NM_001490 |
| | | | | | | | | transferase 1, core 2 (beta- | |
| | | | | | | | | 1,6-N- | |
| | | | | | | | | acetylglucosaminyltransferase) | |
| P01127_B01 | −2.8 | 2.2 | −1.1 | −2.9 | 3.7 | −1 | GDF5 | growth differentiation factors 5 | NM_000557 |
| | | | | | | | | (cartilage-derived | |
| | | | | | | | | morphogenetic protein-1) | |
| P01065_A06 | −1.7 | 1.7 | 1.1 | −1.8 | 1.6 | 1 | GGA3 | golgi associated, gamma | NM_014001 |
| | | | | | | | | adaptin ear containing, ARF | |
| | | | | | | | | binding protein 3 | |
| P01076_H05 | −2 | 2.4 | 1.2 | −2.7 | 1.9 | 1.1 | GM2A | GM2 ganglioside activator | NM_000405 |
| | | | | | | | | protein | |
| P01062_E04 | −2.3 | 1.9 | −1 | −2.1 | 1.9 | −1 | GNPI | glucosamine-6-phosphate | NM_005471 |
| | | | | | | | | isomerase | |
| P01138_C10 | −2.1 | 2.2 | −1.1 | −2 | 1.9 | −1.1 | GNPI | glucosamine-6-phosphate | NM_005471 |
| | | | | | | | | isomerase | |
| P01074_D06 | 3.5 | −4 | −1 | 5.7 | −3.6 | 1.1 | GOLGA4 | golgi autoantigen, golgin | NM_002078 |
| | | | | | | | | subfamily a, 4 | |
| P01083_C04 | −1.1 | 1.2 | 1.2 | −1.8 | 1.5 | 1.1 | GOLPH2 | golgi phosphoprotein 2 | NM_016548 |
| P01125_G10 | 1.8 | −1.9 | −1 | 1.6 | −1.9 | −1.1 | GOLPH4 | golgi phosphoprotein 4 | NM_014498 |
| P01131_F08 | 1.7 | −2.3 | −1.2 | 1.8 | −1.6 | −1.2 | GOT1 | glutamic-oxaloacetic | NM_002079 |
| | | | | | | | | transaminase 1, soluble | |
| | | | | | | | | (aspartate aminotransferase 1) | |
| P01080_A01 | −1.2 | 1.9 | 1.3 | −3.6 | 1.8 | 1.2 | GPM6B | glycoprotein M6B | NM_005278 |
| P01082_E09 | −2.2 | 2.3 | 1.2 | −2.9 | 2.3 | 1 | GPNMB | glycoprotein (transmembrane) | NM_002510 |
| | | | | | | | | nmb | |
| P01087_G08 | −3 | 2.3 | 1 | −4.9 | 4 | −1 | GPNMB | glycoprotein (transmembrane) | NM_002510 |
| | | | | | | | | nmb | |
| P01140_E04 | −1.8 | 1.6 | 1.3 | −2.5 | 1.8 | 1.1 | GPRK5 | G protein-coupled receptor | NM_005308 |
| | | | | | | | | kinase 5 | |
| P01068_E08 | −3.2 | 1.8 | −1.1 | −1.9 | 2.9 | 1.1 | GSTM1 | glutathione S-transferase M1 | NM_000561 |
| P01068_E09 | −1.8 | 1.5 | 1.1 | −1.7 | 2.3 | 1.1 | GSTM3 | glutathione S-transferase M3 | NM_000849 |
| | | | | | | | | (brain) | |
| P01086_A10 | −2.4 | 1.5 | 1.1 | −1.9 | 2.7 | 1.1 | GSTM5 | glutathione S-transferase M5 | NM_000851 |
| P01080_C03 | 1.7 | −1.8 | 1.1 | 2.1 | −1.9 | −1 | GTPBP2 | GTP binding protein 2 | NM_019096 |
| P01108_A05 | −1.2 | 1.5 | 1.2 | −1.9 | 1.7 | 1.1 | GYPC | glycophorin C (Gerbich blood | NM_002101 |
| | | | | | | | | group) | |
| P01121_B02 | −1.6 | 1.2 | −1 | −1.9 | 1.9 | 1.1 | HAGE | DEAD-box protein | NM_018665 |
| P01133_H11 | −1.2 | 1.8 | 1.4 | −2.1 | 1.7 | 1.2 | HAS2 | hyaluronan synthase 2 | NM_005328 |
| P01101_C10 | −1.8 | 1.6 | −1 | −1.4 | 1.5 | −1 | HEBP1 | heme binding protein 1 | NM_015987 |
| P01137_B02 | 1.8 | −1.5 | 1 | 1.6 | −1.3 | 1 | HERPUD1 | homocysteine-inducible, | NM_014685 |
| | | | | | | | | endoplasmic reticulum stress- | |
| | | | | | | | | inducible, ubiquitin-like domain | |
| | | | | | | | | member 1 | |
| P01136_A05 | 2 | −2.1 | 1.1 | 2 | −1.8 | −1 | HERPUD1 | homocysteine-inducible, | NM_014685 |
| | | | | | | | | endoplasmic reticulum stress- | |
| | | | | | | | | inducible, ubiquitin-like domain | |
| | | | | | | | | member 1 | |
| P01083_G12 | 1.6 | −1.1 | 1.1 | 2.2 | −1.5 | 1.2 | HEYL | hairy/enhancer-of-split related | NM_014571 |
| | | | | | | | | with YRPW motif-like | |
| P01126_B01 | −1.3 | 1.4 | 1.1 | −1.8 | 1.8 | −1 | HFL1 | H factor (complement)-like 1 | NM_002113 |
| P01075_H10 | −3.6 | 6.2 | 1.3 | −5.3 | 3.8 | 1.3 | HGF | hepatocyte growth factor | NM_000601 |
| | | | | | | | | (hepapoietin A; scatter factor) | |
| P01110_C10 | 1.9 | −1.6 | 1.3 | 1.3 | −1.2 | 1.1 | HMGCR | 3-hydroxy-3-methylglutaryl- | NM_000859 |
| | | | | | | | | Coenzyme A reductase | |
| P01112_G07 | 2 | −1.7 | 1.3 | −1 | −1.1 | −1 | HMGCS1 | 3-hydroxy-3-methylglutaryl- | NM_002130 |
| | | | | | | | | Coenzyme A synthase 1 | |
| | | | | | | | | (soluble) | |
| P01064_F02 | −2 | 2.6 | 1.1 | −3.1 | 3 | 1 | HNMT | histamine N-methyltransferase | NM_006895 |
| P01078_F05 | −2.1 | 2.4 | 1.2 | −2.1 | 2.6 | 1.1 | HPN | hepsin (transmembrane | NM_002151 |
| | | | | | | | | protease, serine 1) | |
| P01107_H06 | 1.8 | −1.9 | −1.2 | 1.4 | −1.7 | −1.3 | IARS | isoleucine-tRNA synthetase | NM_002161 |
| P01100_C10 | −1.2 | 1.5 | 1.3 | −1.8 | 1.7 | 1.1 | ICOS | inducible T-cell co-stimulator | NM_012092 |
| P01124_A06 | −1.7 | 2 | 1.3 | −1.8 | 1.7 | −1 | ID2 | inhibitor of DNA binding 2, | NM_002166 |
| | | | | | | | | dominant negative helix-loop- | |
| | | | | | | | | helix protein | |
| P01072_H03 | 1.8 | −1.6 | 1.1 | 1.6 | −1.6 | 1.2 | ID4 | inhibitor of DNA binding 4, | NM_001546 |
| | | | | | | | | dominant negative helix-loop- | |
| | | | | | | | | helix protein | |
| P01088_C01 | −2.4 | 2.2 | 1 | −2.5 | 2.2 | −1 | IDH2 | isocitrate dehydrogenase 2 | NM_002168 |
| | | | | | | | | (NADP+), mitochondrial | |
| P01130_F01 | 4.5 | −2.9 | 1.3 | 1.8 | −1.7 | 1 | IGF1 | insulin-like growth factor 1 | NM_000618 |
| | | | | | | | | (somatomedin C) | |
| P01063_D10 | 2.1 | 1 | 1.2 | 3.8 | −1.9 | 1.3 | IGF1 | insulin-like growth factor 1 | NM_000618 |
| | | | | | | | | (somatomedin C) | |
| P00777_D09 | −2.6 | 2.2 | 1.1 | −2.9 | 3.2 | 1.2 | IGFBP4 | insulin-like growth factor | NM_001552 |
| | | | | | | | | binding protein 4 | |
| P01130_B02 | 12.3 | −11.1 | 1.2 | 6.1 | −5.4 | 1.1 | IL11 | interleukin 11 | NM_000641 |
| P01088_D05 | −2 | 2 | 1.2 | −1.8 | 1.4 | 1.1 | IL1B | interleukin 1, beta | NM_000576 |
| P01063_E06 | −3 | 3.3 | 1.1 | −6.7 | 6.1 | 1.1 | IL1R1 | interleukin 1 receptor, type I | NM_000877 |
| P01110_E12 | −1.4 | 2.3 | 1.3 | −2.5 | 1.5 | 1.1 | IL1R1 | interleukin 1 receptor, type I | NM_000877 |
| P01145_A04 | −3 | 2.4 | −1 | −4.2 | 2.7 | −1 | IL6ST | interleukin 6 signal transducer | NM_002184 |
| | | | | | | | | (gp130, oncostatin M receptor) | |
| P01091_B03 | −1.9 | 1.9 | −1 | −1.3 | 1.3 | 1 | IMPA2 | inositol(myo)-1(or 4)- | NM_014214 |
| | | | | | | | | monophosphatase 2 | |
| P01063_E03 | 1.7 | −1.7 | −1.2 | 2.4 | −1.7 | 1.1 | INDO | indoleamine-pyrrole 2,3 | NM_002164 |
| | | | | | | | | dioxygenase | |
| P01082_F07 | 2.1 | −2.6 | −1.1 | 2.2 | −1.5 | 1.2 | INHBA | inhibin, beta A (activin A, | NM_002192 |
| | | | | | | | | activin AB alpha polypeptide) | |
| P01130_D09 | 2.1 | −1.7 | −1 | 1.7 | −1.7 | −1.1 | INPP4B | inositol polyphosphate-4- | NM_003866 |
| | | | | | | | | phosphatase, type II, 105 kDa | |
| P01067_B04 | 2 | −1.7 | 1.2 | 1.6 | −1.3 | 1.1 | INSIG1 | insulin induced gene 1 | NM_005542 |
| P01074_G10 | −1.7 | 1.7 | −1 | −4.7 | 3.1 | −1.1 | IQGAP2 | IQ motif containing GTPase | NM_006633 |
| | | | | | | | | activating protein 2 | |
| P01061_E02 | 2.6 | −2.6 | 1 | 2.4 | −2.5 | −1 | ISGF3G | interferon-stimulated | NM_006084 |
| | | | | | | | | transcription factor 3, gamma | |
| | | | | | | | | 48 kDa | |
| P01140_B08 | 1.8 | −1.7 | 1.2 | 3 | −1.8 | 1 | ITGA11 | integrin, alpha 11 | NM_012211 |
| P01088_C11 | −1.5 | 1.8 | 1.2 | −1.8 | 1.9 | 1.1 | ITGAM | integrin, alpha M (complement | NM_000632 |
| | | | | | | | | component receptor 3, alpha; | |
| | | | | | | | | also known as CD11b (p170), | |
| | | | | | | | | macrophage antigen alpha | |
| | | | | | | | | polypeptide) | |
| P01081_E02 | 2.3 | −1.8 | 1.2 | 2.2 | −2.2 | 1 | JUNB | jun B proto-oncogene | NM_002229 |
| P01072_G01 | 1.6 | −1.5 | 1.2 | 1.9 | −1.6 | 1.1 | JUP | junction plakoglobin | NM_002230 |
| P01079_A01 | −1.9 | 2.1 | −1.1 | −1.5 | 1.5 | −1 | JWA | vitamin A responsive; | NM_006407 |
| | | | | | | | | cytoskeleton related | |
| P01122_A09 | 1.1 | 1.3 | 1.2 | −1.9 | 1.6 | 1.1 | KCNE3 | potassium voltage-gated | NM_005472 |
| | | | | | | | | channel, lsk-related family, | |
| | | | | | | | | member 3 | |
| P01113_F02 | −1.8 | 1.9 | 1.2 | −2.4 | 2.3 | 1.1 | KHDRBS3 | KH domain containing, RNA | NM_006558 |
| | | | | | | | | binding, signal transduction | |
| | | | | | | | | associated 3 | |
| P01074_B01 | −1.6 | 1.2 | 1.1 | −1.9 | 1.7 | 1 | KIAA0102 | KIAA0102 gene product | NM_014752 |
| P01104_A04 | −3.2 | 3.8 | −1 | −3 | 3.4 | −1 | KIAA1049 | KIAA1049 protein | NM_014972 |
| P01120_B02 | −1.6 | 1.5 | 1.1 | −1.8 | 1.7 | 1 | KIF1B | kinesin family member 1B | NM_015074 |
| P01088_C06 | −1.6 | 1.6 | 1.2 | −1.9 | 1.8 | 1 | KRT4 | keratin 4 | NM_002272 |
| P01085_D06 | −1.8 | 1.7 | 1.2 | −3.8 | 4.1 | 1 | LAMA4 | laminin, alpha 4 | NM_002290 |
| P01131_H02 | −1.4 | 1.4 | 1.1 | −2 | 1.9 | −1.1 | LAMC1 | laminin, gamma 1 (formerly | NM_002293 |
| | | | | | | | | LAMB2) | |
| P01131_H10 | −2.4 | 1.8 | −1.1 | −2.1 | 1.5 | 1 | LCN2 | lipocalin 2 (oncogene 24p3) | NM_005564 |
| P01100_H05 | −2.8 | 2.7 | 1.2 | −5 | 2.7 | 1 | LEPR | leptin receptor | NM_002303 |
| P01088_B02 | −2.3 | 2.4 | 1.1 | −2.6 | 2.1 | −1 | LGALS3 | lectin, galactoside-binding, | NM_002306 |
| | | | | | | | | soluble, 3 (galectin 3) | |
| P01081_B11 | −3.5 | 1.3 | 1.1 | −4.6 | 4.4 | 1 | LHFP | lipoma HMGIC fusion partner | NM_005780 |
| P01107_D06 | 2.2 | −2 | −1 | 1.7 | −1.8 | −1.1 | LIMK2 | LIM domain kinase 2 | NM_005569 |
| P01085_G06 | 1.2 | −1.4 | −1.1 | 1.9 | −2.1 | −1 | LMO7 | LIM domain only 7 | NM_005358 |
| P01085_D05 | −2.1 | 2.2 | 1.2 | −3.9 | 3.7 | 1.1 | LOC56270 | hypothetical protein 628 | NM_019613 |
| P01082_E01 | 2.1 | −1.5 | 1.2 | 1.8 | −1.6 | 1.2 | LOX | lysyl oxidase | NM_002317 |
| P01083_H02 | −1.4 | 1.5 | 1.1 | −2 | 2 | 1 | LPHN2 | latrophilin 2 | NM_012302 |
| P01131_D06 | −1.6 | 1.7 | 1.2 | −2.4 | 1.8 | 1.2 | LRP4 | low density lipoprotein | AB011540 |
| | | | | | | | | receptor-related protein 4 | |
| P01072_F03 | 1.8 | −1.2 | −1 | 2.2 | −1.6 | −1 | LTBP2 | latent transforming growth | NM_000428 |
| | | | | | | | | factor beta binding protein 2 | |
| P01088_C04 | −2.3 | 2.3 | 1.1 | −4.4 | 4.7 | 1.1 | LTF | lactotransferrin | NM_002343 |
| P01063_A11 | −2.3 | 2.4 | −1 | −4.8 | 3.9 | −1 | LUM | lumican | NM_002345 |
| P01135_G05 | −2.4 | 2.4 | 1.2 | −1.7 | 1.6 | −1 | LY96 | lymphocyte antigen 96 | NM_015364 |
| P01085_C04 | −2 | 1.8 | 1.2 | −2 | 1.5 | 1 | MADH3 | MAD, mothers against | NM_005902 |
| | | | | | | | | decapentaplegic homolog 3 | |
| | | | | | | | | (Drosophila) | |
| P01091_G10 | 1.8 | −1.4 | 1.2 | 2.2 | −2.1 | 1.2 | MADH7 | MAD, mothers against | NM_005904 |
| | | | | | | | | decapentaplegic homolog 7 | |
| | | | | | | | | (Drosophila) | |
| P01089_C01 | 1.2 | −1.2 | −1 | 1.8 | −1.6 | −1.2 | MAGP2 | Microfibril-associated | NM_003480 |
| | | | | | | | | glycoprotein-2 | |
| P01084_A09 | 1.8 | −1.6 | 1.2 | 1.4 | −1.6 | −1 | MAP3K2 | mitogen-activated protein | NM_006609 |
| | | | | | | | | kinase kinase kinase 2 | |
| P01073_E08 | −2 | 2.4 | −1 | −2.3 | 1.8 | −1 | MAP3K5 | mitogen-activated protein | NM_005923 |
| | | | | | | | | kinase kinase kinase 5 | |
| P01066_F10 | 2 | −2 | 1.1 | 1.9 | −1.7 | 1.1 | MAPK7 | mitogen-activated protein | NM_002749 |
| | | | | | | | | kinase 7 | |
| P01076_B12 | 1.9 | −2.1 | −1.1 | 1.7 | −1.7 | −1.1 | MAPRE2 | microtubule-associated | NM_014268 |
| | | | | | | | | protein, RP/EB family, member 2 | |
| P01134_C04 | 3.1 | −2.1 | 1.1 | 2.8 | −3.3 | −1 | MATN3 | matrilin 3 | NM_002381 |
| P01145_A05 | −1.7 | 1.9 | −1 | −2.6 | 2.1 | −1 | ME1 | malic enzyme 1, NADP(+)- | NM_002395 |
| | | | | | | | | dependent, cytosolic | |
| P01072_D11 | −3.3 | 3.7 | −1 | −3.5 | 3.1 | 1 | MEST | mesoderm specific transcript | NM_002402 |
| | | | | | | | | homolog (mouse) | |
| P01121_F04 | −1.9 | 2.1 | 1.3 | −2.1 | 1.8 | 1 | MGC1203 | hypothetical protein MGC1203 | NM_024296 |
| P01068_F12 | −2.9 | 2.8 | 1.1 | −2.6 | 2.4 | −1 | MGST1 | microsomal glutathione S- | NM_020300 |
| | | | | | | | | transferase 1 | |
| P01091_B06 | −1.8 | 1.6 | −1 | −1.5 | 1.6 | −1.1 | MGST2 | microsomal glutathione S- | NM_002413 |
| | | | | | | | | transferase 2 | |
| P01099_H09 | −2.4 | 2.3 | 1.1 | −2.4 | 2 | 1.2 | MID1 | midline 1 (Opitz/BBB | NM_000381 |
| | | | | | | | | syndrome) | |
| P01062_H05 | −1.4 | 2.2 | 1.3 | −2.4 | 2.4 | 1.3 | MME | membrane metallo- | NM_000902 |
| | | | | | | | | endopeptidase (neutral | |
| | | | | | | | | endopeptidase, | |
| | | | | | | | | enkephalinase, CALLA, CD10) | |
| P01125_H08 | 1 | 1 | 1.1 | 2.6 | −2.1 | −1 | MMP11 | matrix metalloproteinase 11 | NM_005940 |
| | | | | | | | | (stromelysin 3) | |
| P01072_D02 | 2.8 | −2.6 | −1.3 | 1.7 | −2 | −1.2 | MTHFD2 | methylene tetrahydrofolate | NM_006636 |
| | | | | | | | | dehydrogenase (NAD+ | |
| | | | | | | | | dependent), | |
| | | | | | | | | methenyltetrahydrofolate | |
| | | | | | | | | cyclohydrolase | |
| P01125_A10 | −1.6 | 1.6 | 1.2 | −1.8 | 1.5 | 1.1 | MTMR4 | myotubularin related protein 4 | NM_004687 |
| P01130_C09 | 1.9 | −1.7 | 1.3 | 1.1 | −1.1 | 1.1 | MUCDHL | mucin and cadherin-like | NM_017717 |
| P01102_A12 | 1.4 | −1.3 | 1.2 | 2.5 | −1.6 | 1.1 | MVK | mevalonate kinase (mevalonic | NM_000431 |
| | | | | | | | | aciduria) | |
| P01133_F05 | 1.9 | −1.8 | 1 | 1.4 | −1.4 | −1.1 | MYH9 | myosin, heavy polypeptide 9, | NM_002473 |
| | | | | | | | | non-muscle | |
| P01100_B07 | −2 | 2.4 | 1.1 | −5.5 | 2.6 | −1.1 | MYOZ2 | myozenin 2 | NM_016599 |
| P01072_C06 | −1.6 | 1.6 | 1 | −2.6 | 2.6 | 1.1 | NCK1 | NCK adaptor protein 1 | NM_006153 |
| P01086_B12 | −1.2 | 1.4 | −1 | −1.8 | 1.6 | −1.1 | NCOA3 | nuclear receptor coactivator 3 | NM_006534 |
| P01135_C12 | 3.3 | −3 | 1.3 | 3.1 | −2.1 | 1.2 | NEDD9 | neural precursor cell | NM_006403 |
| | | | | | | | | expressed, developmentally | |
| | | | | | | | | down-regulated 9 | |
| P01112_A08 | 2.5 | −2.1 | 1.2 | 1.7 | −1.8 | 1.1 | NET-6 | transmembrane 4 superfamily | NM_014399 |
| | | | | | | | | member tetraspan NET-6 | |
| P01103_E02 | −1.7 | 2.1 | 1.2 | −2.5 | 2.1 | −1 | NFIA | nuclear factor I/A | AL096888 |
| P01073_E06 | −1.9 | 1.9 | 1 | −2.1 | 1.8 | −1.1 | NFIB | nuclear factor I/B | NM_005596 |
| P01064_C02 | −1.9 | 2 | 1.2 | −3.3 | 2.5 | 1 | NID2 | nidogen 2 (osteonidogen) | NM_007361 |
| P01131_E08 | 2.3 | −1.6 | 1.3 | 5.1 | −3 | 1.3 | NINJ2 | ninjurin 2 | NM_016533 |
| P01072_D01 | 2.2 | −2.2 | 1.1 | 2.2 | −2.1 | −1 | NK4 | natural killer cell transcript 4 | NM_004221 |
| P01121_G06 | −2.2 | 2.1 | −1.1 | −2.5 | 2.2 | −1.1 | NOL3 | nucleolar protein 3 (apoptosis | NM_003946 |
| | | | | | | | | repressor with CARD domain) | |
| P01104_C08 | 6.9 | −6.1 | 1.1 | 5.8 | −5.8 | 1.1 | NOX4 | NADPH oxidase 4 | NM_016931 |
| P01107_D11 | −1.7 | 1.6 | −1 | −1.8 | 1.8 | 1 | NPC2 | Niemann-Pick disease, type | NM_006432 |
| | | | | | | | | C2 | |
| P01132_G06 | 2.4 | −2 | 1.3 | 1.5 | −1.6 | 1.1 | NPR3 | natriuretic peptide receptor | NM_000908 |
| | | | | | | | | C/guanylate cyclase C | |
| | | | | | | | | (atrionatriuretic peptide | |
| | | | | | | | | receptor C) | |
| P01096_F08 | −1.5 | 1.6 | 1.2 | −2.1 | 2 | 1.1 | NPTX2 | neuronal pentraxin II | NM_002523 |
| P01126_E07 | −1.5 | 2 | 1.2 | −2 | 1.7 | 1.1 | NR2F2 | nuclear receptor subfamily 2, | NM_021005 |
| | | | | | | | | group F, member 2 | |
| P01064_G11 | −1.5 | 1.6 | 1 | −2.1 | 1.5 | −1 | NRCAM | neuronal cell adhesion | NM_005010 |
| | | | | | | | | molecule | |
| P01097_E11 | 1.9 | −1.8 | 1.1 | 1.6 | −1.8 | −1 | NS1-BP | NS1-binding protein | NM_006469 |
| P01103_C04 | 2.4 | −2.2 | −1 | 1.3 | −1.5 | −1 | NUDT3 | nudix (nucleoside diphosphate | NM_006703 |
| | | | | | | | | linked moiety X)-type motif 3 | |
| P01072_B11 | 2.6 | −2.6 | −1.1 | 2.3 | −2.3 | −1.2 | ODC1 | omithine decarboxylase 1 | NM_002539 |
| P01082_E10 | −1.4 | 2 | 1.3 | −5.7 | 1.9 | 1.2 | OGN | osteoglycin (osteoinductive | NM_014057 |
| | | | | | | | | factor, mimecan) | |
| P01119_G07 | −2.1 | 2.2 | 1.1 | −2.2 | 1.6 | −1 | OSBPL1A | oxysterol binding protein-like | NM_018030 |
| | | | | | | | | 1A | |
| P01075_F01 | 2.3 | −1.6 | −1 | 3.9 | −3.7 | −1 | OSF-2 | osteoblast specific factor 2 | NM_006475 |
| | | | | | | | | (fasciclin I-like) | |
| P01129_A10 | 2.2 | −1.6 | −1 | 4.1 | −3.6 | −1 | OSF-2 | osteoblast specific factor 2 | NM_006475 |
| | | | | | | | | (fasciclin I-like) | |
| P01126_B11 | −2 | 1.5 | −1.2 | 1.1 | 1.7 | 1.2 | OXA1L | oxidase (cytochrome c) | NM_005015 |
| | | | | | | | | assembly 1-like | |
| P01071_D09 | −1.9 | 1.6 | −1.1 | 1.1 | 1.7 | 1.3 | OXA1L | oxidase (cytochrome c) | NM_005015 |
| | | | | | | | | assembly 1-like | |
| P01085_C08 | −1.3 | 1.3 | 1.1 | −2.2 | 1.6 | −1 | OXTR | oxytocin receptor | NM_000916 |
| P01125_D04 | 2.1 | −1.7 | 1.2 | 4.2 | −2.3 | 1.3 | PACE4 | paired basic amino acid | NM_002570 |
| | | | | | | | | cleaving system 4 | |
| P01090_D03 | −1.4 | 1.2 | −1 | −2.4 | 1.8 | −1.2 | PARG1 | PTPL1-associated RhoGAP 1 | NM_004815 |
| P01122_G06 | 2.6 | −2.4 | 1.1 | 1.9 | −2 | −1 | PAWR | PRKC, apoptosis, WT1, | NM_002583 |
| | | | | | | | | regulator | |
| P01120_F04 | −1.8 | 2.3 | 1.3 | −2 | 1.7 | 1.1 | PBF | papillomavirus regulatory | NM_018660 |
| | | | | | | | | factor PRF-1 | |
| P01071_G08 | −2 | 1.5 | −1 | −1.4 | 1.7 | 1 | PBP | prostatic binding protein | NM_002567 |
| P01064_A09 | 1.1 | −1.2 | 1.2 | 1.9 | −1.5 | 1.2 | PCDH1 | protocadherin 1 (cadherin-like | NM_002587 |
| | | | | | | | | 1) | |
| P01066_G05 | −1.4 | 1.6 | 1.3 | −3.2 | 2.4 | 1.2 | PDE1A | phosphodiesterase 1A, | NM_005019 |
| | | | | | | | | calmodulin-dependent | |
| P01128_B03 | 1.8 | −1.7 | 1.1 | −1.1 | −1 | −1 | PDE5A | phosphodiesterase 5A, cGMP- | NM_001083 |
| | | | | | | | | specific | |
| P01087_E02 | 3.4 | −2.4 | 1.1 | 3 | −3.7 | −1 | PDGFA | platelet-derived growth factor | NM_002607 |
| | | | | | | | | alpha polypeptide | |
| P01081_F07 | −2.3 | 2.1 | 1.1 | −2.2 | 2.1 | 1.1 | PDGFRA | platelet-derived growth factor | NM_006206 |
| | | | | | | | | receptor, alpha polypeptide | |
| P01142_D01 | −1.1 | −1.8 | 1.2 | −2.2 | 1.9 | 1.2 | PDGFRL | platelet-derived growth factor | NM_006207 |
| | | | | | | | | receptor-like | |
| P01064_G02 | 1.3 | −1.1 | 1.3 | 2.3 | −2 | 1.2 | PDGFRL | platelet-derived growth factor | NM_006207 |
| | | | | | | | | receptor-like | |
| P01137_F04 | −1.8 | 2 | 1.1 | −2 | 1.4 | 1.1 | PDP | pyruvate dehydrogenase | NM_018444 |
| | | | | | | | | phosphatase | |
| P01071_H07 | 1.8 | −1.9 | 1 | 1.3 | −1 | 1.1 | PFKP | phosphofructokinase, platelet | NM_002627 |
| P01064_H07 | −1.8 | 1.7 | 1 | −1.6 | 1.5 | 1 | PHF3 | PHD finger protein 3 | NM_015153 |
| P01131_G12 | 1.2 | −1 | 1.2 | 1.8 | −1.7 | 1.2 | PIGB | phosphatidylinositol glycan, | NM_004855 |
| | | | | | | | | class B | |
| P01074_H07 | −1.8 | 1.9 | 1.2 | −1.9 | 1.6 | 1 | PIK3R1 | phosphoinositide-3-kinase, | AF279367 |
| | | | | | | | | regulatory subunit, polypeptide | |
| | | | | | | | | 1 (p85 alpha) | |
| P01068_A02 | −2.4 | 1.7 | −1.1 | −1.4 | 2.1 | 1 | PIR | Pirin | NM_003662 |
| P01112_H01 | 1.8 | −1.6 | 1.4 | 1.3 | −1.4 | −1 | PIST | PDZ/coiled-coil domain | NM_020399 |
| | | | | | | | | binding partner for the rho- | |
| | | | | | | | | family GTPase TC10 | |
| P01118_H09 | −2.4 | 2.1 | −1 | −1.8 | 1.9 | 1 | PITPNM | phosphatidylinositol transfer | NM_004910 |
| | | | | | | | | protein, membrane-associated | |
| P01110_G02 | −1.3 | 1.6 | 1.3 | −4 | 2 | 1 | PKIB | protein kinase (cAMP- | NM_032471 |
| | | | | | | | | dependent, catalytic) inhibitor | |
| | | | | | | | | beta | |
| P01146_A11 | 1.4 | −1.5 | −1 | 1.8 | −1.9 | −1 | PLA2G4C | phospholipase A2, group IVC | NM_003706 |
| | | | | | | | | (cytosolic, calcium- | |
| | | | | | | | | independent) | |
| P01124_G10 | 3 | −2.5 | 1 | 2.5 | −3.2 | −1.2 | PLA2R1 | phospholipase A2 receptor 1, | NM_007366 |
| | | | | | | | | 180 kDa | |
| P01070_G08 | 1.8 | −1.7 | −1 | 1.9 | −2.3 | −1.1 | PLAU | plasminogen activator, | NM_002658 |
| | | | | | | | | urokinase | |
| P01064_F01 | −1.8 | 2.3 | 1.2 | −1.7 | 1.9 | 1.2 | PLCL1 | phospholipase C-like 1 | NM_006226 |
| P01118_E04 | 2.4 | −1.8 | 1.3 | 2.3 | −1.9 | 1.1 | PLEK2 | pleckstrin 2 | NM_016445 |
| P01072_A03 | 5.2 | −5.1 | 1.3 | 2.1 | −1.6 | 1.2 | PLN | phospholamban | NM_002667 |
| P01084_A08 | 2.8 | −2.2 | 1.1 | 1.9 | −1.8 | 1.1 | PLOD2 | procollagen-lysine, 2- | NM_000935 |
| | | | | | | | | oxoglutarate 5-dioxygenase | |
| | | | | | | | | (lysine hydroxylase) 2 | |
| P01063_E04 | 1.6 | −1.7 | −1.2 | 2.4 | −1.6 | 1.1 | PLP2 | proteolipid protein 2 (colonic | NM_002668 |
| | | | | | | | | epithelium-enriched) | |
| P01130_B04 | −3.6 | 4.3 | 1 | −5.6 | 5.1 | 1.1 | PMP2 | peripheral myelin protein 2 | NM_002677 |
| P01131_C08 | −2.2 | 1.6 | 1.1 | −3.4 | 2.3 | 1.1 | PNUTL2 | peanut-like 2 (Drosophila) | NM_004574 |
| P01106_F02 | 1.5 | −1.3 | 1.2 | 1.8 | −1.7 | 1.1 | PODXL | podocalyxin-like | NM_005397 |
| P01074_B08 | 2.8 | −1.8 | 1.2 | 2.2 | −2.8 | 1.1 | POLD3 | polymerase (DNA directed), | BC020587 |
| | | | | | | | | delta 3 | |
| P01080_A04 | −1.3 | 1.4 | 1 | −1.8 | 1.5 | 1.1 | PP | pyrophosphatase (inorganic) | NM_021129 |
| P01123_E01 | −2.9 | 3.2 | 1.3 | −3.1 | 2.8 | 1.3 | PPAP2B | phosphatidic acid phosphatase | NM_003713 |
| | | | | | | | | type 2B | |
| P01064_B12 | −1.5 | 1.7 | 1.2 | −2.2 | 1.5 | −1 | PPARG | peroxisome proliferative | NM_005037 |
| | | | | | | | | activated receptor, gamma | |
| P01136_D03 | −5.4 | 3.3 | 1.1 | −5.3 | 4.2 | −1 | PPL | periplakin | NM_002705 |
| P01131_H04 | 1.2 | −1.4 | −1.2 | 2 | −1.6 | −1.2 | PPP2R4 | protein phosphatase 2A, | NM_021131 |
| | | | | | | | | regulatory subunit B′ (PR 53) | |
| P01087_D04 | −1.2 | 1.3 | −1.1 | −1.9 | 1.5 | −1.1 | PRKCM | protein kinase C, mu | NM_002742 |
| P01128_H07 | 2.3 | −2.2 | 1.3 | 1.4 | −1.4 | 1.1 | PRPS1 | phosphoribosyl pyrophosphate | NM_002764 |
| | | | | | | | | synthetase 1 | |
| P01062_F06 | −1.6 | 1.5 | 1.1 | −4.4 | 3.6 | 1 | PSG1 | pregnancy specific beta-1- | NM_006905 |
| | | | | | | | | glycoprotein 1 | |
| P01133_G04 | −2 | 1.9 | 1.2 | −5.5 | 4.8 | −1 | PSG1 | pregnancy specific beta-1- | NM_006905 |
| | | | | | | | | glycoprotein 1 | |
| P01131_G08 | −1.4 | 1.4 | 1.2 | −2.6 | 2.6 | 1.1 | PSG11 | pregnancy specific beta-1- | NM_002785 |
| | | | | | | | | glycoprotein 11 | |
| P01141_B07 | −1.4 | 1.8 | 1.3 | −4.1 | 4 | 1.1 | PSG4 | pregnancy specific beta-1- | NM_002780 |
| | | | | | | | | glycoprotein 4 | |
| P01079_F07 | −1.5 | 1.5 | 1.1 | −2 | 1.5 | −1 | PTGER4 | prostaglandin E receptor 4 | NM_000958 |
| | | | | | | | | (subtype EP4) | |
| P01131_C07 | −2.8 | 1.7 | −1.1 | −2.2 | 1.8 | −1.1 | PTGIS | prostaglandin I2 (prostacyclin) | NM_000961 |
| | | | | | | | | synthase | |
| P01102_D10 | 2.3 | −2.8 | −1.1 | 1.1 | −1.2 | −1.1 | PTGS1 | prostaglandin-endoperoxide | NM_000962 |
| | | | | | | | | synthase 1 (prostaglandin G/H | |
| | | | | | | | | synthase and cyclooxygenase) | |
| P01087_D05 | 3 | −2.6 | 1.1 | 1.3 | −1.2 | −1.1 | PTGS2 | prostaglandin-endoperoxide | NM_000963 |
| | | | | | | | | synthase 2 (prostaglandin G/H | |
| | | | | | | | | synthase and cyclooxygenase) | |
| P01106_G06 | 1.8 | −1.5 | 1.2 | 3.1 | −1.5 | 1.1 | PTHLH | parathyroid hormone-like | NM_002820 |
| | | | | | | | | hormone | |
| P01071_G12 | −1.9 | 1.4 | −1 | −3.7 | 3.6 | −1.1 | PTN | pleiotrophin (heparin binding | NM_002825 |
| | | | | | | | | growth factor 8, neurite | |
| | | | | | | | | growth-promoting factor 1) | |
| P01128_H08 | −2.3 | 2.4 | 1.1 | −1.5 | 1.3 | −1 | PTTG1 | pituitary tumor-transforming 1 | NM_004219 |
| P01095_A03 | −2.4 | 2.4 | 1.2 | −1.4 | 1.2 | 1 | PTTG1 | pituitary tumor-transforming 1 | NM_004219 |
| P01097_G06 | −1.7 | 1.7 | −1 | −2.2 | 1.6 | −1 | PUS1 | pseudouridylate synthase 1 | NM_025215 |
| P01076_C04 | 2.5 | −1.7 | 1.2 | 3.1 | −2.6 | 1.2 | QPCT | glutaminyl-peptide | NM_012413 |
| | | | | | | | | cyclotransferase (glutaminyl | |
| | | | | | | | | cyclase) | |
| P01129_C05 | −2.1 | 1.8 | −1 | −1.5 | 2.1 | 1.2 | RAB13 | RAB13, member RAS | NM_002870 |
| | | | | | | | | oncogene family | |
| P01115_G01 | −1.8 | 1.5 | 1.1 | −1.6 | 2.2 | 1.2 | RAB13 | RAB13, member RAS | NM_002870 |
| | | | | | | | | oncogene family | |
| P01110_E09 | 1.4 | −1.2 | 1.2 | 1.8 | −1.6 | 1 | RAI | RelA-associated inhibitor | NM_006663 |
| P01100_E02 | −1.5 | 1.5 | 1.1 | −3.3 | 2.7 | 1 | RAI3 | retinoic acid induced 3 | NM_003979 |
| P01082_A01 | −2.4 | 1.8 | 1.1 | −2.6 | 2.3 | 1.1 | RARRES3 | retinoic acid receptor | NM_004585 |
| | | | | | | | | responder (tazarotene | |
| | | | | | | | | induced) 3 | |
| P01117_H10 | −1.8 | 1.6 | 1.1 | −2.5 | 2 | 1 | RASSF5 | Ras association (RalGDS/AF- | NM_031437 |
| | | | | | | | | 6) domain family 5 | |
| P01108_C07 | 1.4 | −1.4 | 1.1 | 2.5 | −1.9 | 1.2 | RBP1 | retinol binding protein 1, | NM_002899 |
| | | | | | | | | cellular | |
| P01136_C04 | 2.6 | −1.8 | 1.1 | 2.3 | −1.6 | 1.2 | RGS2 | regulator of G-protein | NM_002923 |
| | | | | | | | | signalling 2, 24 kDa | |
| P01145_A10 | −1.2 | 1.2 | −1.1 | −2 | 1.6 | −1.1 | RGS4 | regulator of G-protein | NM_005613 |
| | | | | | | | | signalling 4 | |
| P01090_D02 | −1.3 | 1.2 | −1.1 | −3 | 1.9 | −1.3 | RGS4 | regulator of G-protein | NM_005613 |
| | | | | | | | | signalling 4 | |
| P01081_H10 | −2.2 | 1.6 | 1 | −6.7 | 4.7 | −1 | RGS5 | regulator of G-protein | NM_003617 |
| | | | | | | | | signalling 5 | |
| P01071_E04 | −1.9 | 1.4 | −1.1 | −3.8 | 3.2 | −1.1 | RNASE1 | ribonuclease, RNase A family, | NM_002933 |
| | | | | | | | | 1 (pancreatic) | |
| P01088_G09 | −1 | 1 | 1 | −1.8 | 1.8 | −1.1 | RPL5 | ribosomal protein L5 | NM_000969 |
| P01127_E10 | 1.8 | −1.6 | 1.1 | 1.7 | −1.5 | −1 | RRAS | related RAS viral (r-ras) | NM_006270 |
| | | | | | | | | oncogene homolog | |
| P01122_B03 | −2 | 2 | 1.1 | −2.4 | 3.1 | 1.2 | RRP4 | homolog of Yeast RRP4 | NM_014285 |
| | | | | | | | | (ribosomal RNA processing 4), | |
| | | | | | | | | 3′-5′-exoribonuclease | |
| P01104_D09 | 2.1 | −1.7 | 1.1 | 2 | −1.8 | 1 | RTP801 | HIF-1 responsive RTP801 | NM_019058 |
| P01121_G04 | 2.1 | −1.8 | 1.1 | 4.1 | −3.2 | 1.1 | RUVBL2 | RuvB-like 2 (E. coli) | NM_006666 |
| P01087_B06 | −1.4 | 1 | −1.2 | −1.9 | 2.4 | −1.2 | S100A10 | S100 calcium binding protein | NM_002966 |
| | | | | | | | | A10 (annexin II ligand, | |
| | | | | | | | | calpactin I, light polypeptide | |
| | | | | | | | | (p11)) | |
| P01064_F10 | 1.5 | −1.5 | −1.3 | 1.8 | −1.5 | −1.2 | S100A11 | S100 calcium binding protein | NM_005620 |
| | | | | | | | | A11 (calgizzarin) | |
| P00777_A05 | −1.9 | 1.7 | 1.1 | −2.3 | 2.4 | 1.1 | S100A4 | S100 calcium binding protein | NM_002961 |
| | | | | | | | | A4 (calcium protein, | |
| | | | | | | | | calvasculin, metastasin, | |
| | | | | | | | | murine placental homolog) | |
| P00777_A06 | −1.9 | 1.8 | 1.1 | −2.6 | 2.7 | 1.1 | S100A4 | S100 calcium binding protein | NM_002961 |
| | | | | | | | | A4 (calcium protein, | |
| | | | | | | | | calvasculin, metastasin, | |
| | | | | | | | | murine placental homolog) | |
| P01143_A11 | −1.7 | 1.7 | 1.1 | −2.4 | 2.4 | 1.1 | S100A4 | S100 calcium binding protein | NM_002961 |
| | | | | | | | | A4 (calcium protein, | |
| | | | | | | | | calvasculin, metastasin, | |
| | | | | | | | | murine placental homolog) | |
| P01141_F03 | 1.5 | −1.2 | 1.3 | 3.9 | −1.7 | 1.3 | SAA2 | serum amyloid A2 | NM_030754 |
| P01061_F04 | −3.1 | 4 | 1.3 | −2.2 | 2.8 | 1.3 | SAT | spermidine/spermine N1- | NM_002970 |
| | | | | | | | | acetyltransferase | |
| P01124_B03 | −2.9 | 3.7 | 1.4 | −2.1 | 2.5 | 1.4 | SAT | spermidine/spermine N1- | NM_002970 |
| | | | | | | | | acetyltransferase | |
| P01140_G05 | 2 | −2.1 | 1.1 | 1.3 | −1.3 | −1 | SC5DL | sterol-C5-desaturase (ERG3 | NM_006918 |
| | | | | | | | | delta-5-desaturase homolog, | |
| | | | | | | | | fungal)-like | |
| P01066_H04 | 4.1 | −2.7 | 1.2 | 3 | −2 | 1.2 | SCD | stearoyl-CoA desaturase | NM_005063 |
| | | | | | | | | (delta-9-desaturase) | |
| P01140_D11 | 4.7 | −3.8 | 1.2 | 3.5 | −2.4 | 1 | SCD | stearoyl-CoA desaturase | NM_005063 |
| | | | | | | | | (delta-9-desaturase) | |
| P01119_B12 | −1.6 | 1.9 | 1.2 | −3.9 | 2.3 | 1.1 | SCDGF-B | spinal cord-derived growth | NM_025208 |
| | | | | | | | | factor-B | |
| P01087_A04 | −1.1 | 1.3 | 1.2 | −4 | 2 | 1.2 | SCG2 | secretogranin II (chromogranin | NM_003469 |
| | | | | | | | | C) | |
| P01096_B12 | 2.6 | −1.9 | 1.2 | 2.8 | −2.5 | −1 | SCRG1 | scrapie responsive protein 1 | NM_007281 |
| P01071_B04 | −1.7 | 1.7 | −1.1 | −2.6 | 2.3 | 1 | SDC4 | syndecan 4 (amphiglycan, | NM_002999 |
| | | | | | | | | ryudocan) | |
| P01063_H09 | −1.8 | 1.7 | 1 | −1.8 | 1.6 | −1 | SDCBP | syndecan binding protein | NM_005625 |
| | | | | | | | | (syntenin) | |
| P01076_C05 | 1.8 | −1.5 | 1.2 | 1 | −1.2 | −1 | SEC23A | Sec23 homolog A (S. cerevisiae) | NM_006364 |
| P01096_G04 | −3.6 | 2.5 | −1 | −3 | 6 | 1.3 | SELENBP1 | selenium binding protein 1 | NM_003944 |
| P01119_G09 | −3.2 | 2.4 | 1.1 | −2.5 | 5.8 | 1.4 | SELENBP1 | selenium binding protein 1 | NM_003944 |
| P01076_B03 | −1.6 | 1.4 | −1 | −2 | 1.5 | −1.1 | SEPP1 | selenoprotein P, plasma, 1 | NM_005410 |
| P01062_D11 | 3 | −2.9 | −1 | 4.3 | −3.3 | −1 | SERPINE1 | serine (or cysteine) proteinase | NM_000602 |
| | | | | | | | | inhibitor, clade E (nexin, | |
| | | | | | | | | plasminogen activator inhibitor | |
| | | | | | | | | type 1), member 1 | |
| P01090_H11 | −1.3 | 1.2 | 1.2 | −1.9 | 2.1 | 1.3 | SFRP1 | secreted frizzled-related | NM_003012 |
| | | | | | | | | protein 1 | |
| P01078_F01 | −1.8 | 2.4 | −1.1 | −1.6 | 1.6 | 1.1 | SFRP4 | secreted frizzled-related | NM_003014 |
| | | | | | | | | protein 4 | |
| P01087_A06 | −2.9 | 1.9 | −1.2 | −3 | 2.2 | −1.3 | SGNE1 | secretory granule, | NM_003020 |
| | | | | | | | | neuroendocrine protein 1 (7B2 | |
| | | | | | | | | protein) | |
| P01106_G05 | 1.8 | −1.7 | 1.3 | 2.9 | −2.2 | 1.2 | SKIL | SKI-like | NM_005414 |
| P01102_A06 | −1.8 | 2 | 1.3 | −3.2 | 2.8 | 1.3 | SLC11A3 | solute carrier family 11 | NM_014585 |
| | | | | | | | | (proton-coupled divalent metalion | |
| | | | | | | | | transporters), member 3 | |
| P01105_A03 | 1.9 | −1.7 | 1.1 | 1.5 | −1.4 | −1 | SLC1A4 | solute carrier family 1 | NM_003038 |
| | | | | | | | | (glutamate/neutral amino acid | |
| | | | | | | | | transporter), member 4 | |
| P01143_D11 | −2.7 | 2.5 | 1.3 | −2.1 | 2.8 | 1.1 | SLC25A11 | solute carrier family 25 | NM_003562 |
| | | | | | | | | (mitochondrial carrier; | |
| | | | | | | | | oxoglutarate carrier), member | |
| | | | | | | | | 11 | |
| P01111_H03 | 1.8 | −1.7 | 1 | 1.9 | −2 | −1 | SLC7A11 | solute carrier family 7, | NM_014331 |
| | | | | | | | | (cationic amino acid | |
| | | | | | | | | transporter, y+ system) | |
| | | | | | | | | member 11 | |
| P01138_A08 | 3 | −2.9 | −1 | 2.3 | −2.4 | −1.1 | SLC7A5 | solute carrier family 7 (cationic | NM_003486 |
| | | | | | | | | amino acid transporter, y+ | |
| | | | | | | | | system), member 5 | |
| P01088_E10 | 3.1 | −2.7 | 1.1 | 2.6 | −2.3 | 1 | SLC7A5 | solute carrier family 7 (cationic | NM_003486 |
| | | | | | | | | amino acid transporter, y+ | |
| | | | | | | | | system), member 5 | |
| P01112_E05 | −1.6 | 1.3 | 1 | −3 | 2.2 | −1 | SLIT3 | slit homolog 3 (Drosophila) | NM_003062 |
| P01136_F07 | −1.1 | 1.4 | 1.2 | −2.1 | 1.9 | 1.1 | SLIT3 | slit homolog 3 (Drosophila) | NM_003062 |
| P01079_G03 | −3.1 | 3.4 | −1 | −3.9 | 3.5 | −1 | SNAI2 | snail homolog 2 (Drosophila) | NM_003068 |
| P01140_F07 | 2.9 | −2.6 | 1.1 | 2.2 | −2.5 | 1.1 | SNF1LK | SNF1-like kinase | |
| P01083_A04 | −3.2 | 3.2 | 1 | −9.3 | 7 | −1.1 | SNK | serum-inducible kinase | NM_006622 |
| P01085_F06 | −1.2 | 1.2 | 1.1 | −2.6 | 1.7 | 1.1 | SOD3 | superoxide dismutase 3, | NM_003102 |
| | | | | | | | | extracellular | |
| P01074_H12 | 1 | 1.1 | 1.1 | −2.6 | 1.5 | 1.1 | SPINT2 | serine protease inhibitor, | NM_021102 |
| | | | | | | | | Kunitz type, 2 | |
| P01108_B02 | −2.5 | 2.6 | 1.2 | −4.2 | 2.2 | −1 | SPRY1 | sprouty homolog 1, antagonist | AF041037 |
| | | | | | | | | of FGF signaling (Drosophila) | |
| P01095_F04 | −2.6 | 2 | −1.1 | −1.8 | 1.8 | −1.1 | SQRDL | sulfide quinone reductase-like | NM_021199 |
| | | | | | | | | (yeast) | |
| P01128_E07 | 1.9 | −2 | 1 | 2.6 | −2.7 | −1 | SRPUL | sushi-repeat protein | NM_014467 |
| P01073_B02 | −1.7 | 1.7 | 1.2 | −2.5 | 1.9 | −1 | SRPX | sushi-repeat-containing | NM_006307 |
| | | | | | | | | protein, X chromosome | |
| P01104_F12 | −2.1 | 2.5 | 1.2 | −2.2 | 2.3 | −1.1 | SSBP2 | single-stranded DNA binding | NM_012446 |
| | | | | | | | | protein 2 | |
| P01069_C06 | 1.9 | −1.3 | −1 | 2.7 | −2.6 | −1 | SSR1 | signal sequence receptor, | NM_003144 |
| | | | | | | | | alpha (translocon-associated | |
| | | | | | | | | protein alpha) | |
| P01130_F10 | −1.3 | 1.6 | 1.1 | −2.3 | 2.3 | −1.1 | STC1 | stanniocalcin 1 | NM_003155 |
| P01130_B11 | 2.1 | −2 | −1 | 1.7 | −1.8 | −1.1 | STCH | stress 70 protein chaperone, | NM_006948 |
| | | | | | | | | microsome-associated, 60 kDa | |
| P01074_E03 | 1.7 | −1.3 | −1 | −1.9 | 1.5 | −1.1 | STE | sulfotransferase, estrogen- | NM_005420 |
| | | | | | | | | preferring | |
| P01127_G01 | −1.4 | 1.5 | 1.2 | −1.9 | 1.5 | 1.2 | STK17B | serine/threonine kinase 17b | NM_004226 |
| | | | | | | | | (apoptosis-inducing) | |
| P01125_C11 | −2 | 1.6 | −1 | −2.2 | 2.1 | −1 | STK25 | serine/threonine kinase 25 | NM_006374 |
| | | | | | | | | (STE20 homolog, yeast) | |
| P01076_D03 | −2.7 | 2.9 | 1.1 | −2.1 | 1.8 | 1 | STK38 | serine/threonine kinase 38 | NM_007271 |
| P01105_E03 | −2.8 | 2.7 | −1.1 | −2.7 | 2.5 | −1.1 | STMN1 | stathmin 1/oncoprotein 18 | NM_005563 |
| P01069_A08 | −1.5 | 1.5 | 1.1 | −1.8 | 1.5 | 1 | STOM | stomatin | NM_004099 |
| P01102_E10 | −1.5 | 1.6 | 1.1 | −2.3 | 1.5 | 1 | SVIL | supervillin | NM_003174 |
| P01062_H06 | −1.5 | 2.1 | 1.2 | −2.9 | 2.7 | 1.2 | TACSTD2 | tumor-associated calcium | NM_002353 |
| | | | | | | | | signal transducer 2 | |
| P01098_E05 | 1.9 | −1.8 | 1.2 | 1.2 | −1.3 | 1.1 | TAF13 | TAF13 RNA polymerase II, | NM_005645 |
| | | | | | | | | TATA box binding protein | |
| | | | | | | | | (TBP)-associated factor, | |
| | | | | | | | | 18 kDa | |
| P01101_B02 | −1.9 | 1.4 | 1.1 | −2 | 1.8 | 1 | TCF7L1 | transcription factor 7-like 1 (T- | NM_031283 |
| | | | | | | | | cell specific, HMG-box) | |
| P01061_C01 | −1.7 | 1.6 | 1.3 | −2 | 2.4 | 1.1 | TF | transferrin | NM_001063 |
| P01144_C03 | −3.4 | 3.6 | 1.2 | −4 | 2.9 | 1.1 | TFPI | tissue factor pathway inhibitor | NM_006287 |
| | | | | | | | | (lipoprotein-associated | |
| | | | | | | | | coagulation inhibitor) | |
| P01071_A04 | −1.4 | 1.6 | 1.4 | −2.3 | 2.3 | 1.1 | TFPI2 | tissue factor pathway inhibitor 2 | NM_006528 |
| P01085_B12 | −1.4 | 1.4 | 1.2 | −2.1 | 1.7 | 1.1 | TGFB2 | transforming growth factor, | NM_003238 |
| | | | | | | | | beta 2 | |
| P01061_C08 | −3.5 | 3.8 | 1.2 | −4.7 | 4 | 1.2 | TGFBR3 | transforming growth factor, | NM_003243 |
| | | | | | | | | beta receptor III (betaglycan, | |
| | | | | | | | | 300 kDa) | |
| P01078_B04 | 1.8 | −1.7 | −1 | 1.8 | −1.9 | −1.2 | THBS2 | thrombospondin 2 | NM_003247 |
| P01124_G04 | 2.8 | −2.5 | −1 | 2.4 | −3.1 | −1.3 | TIMP3 | tissue inhibitor of | NM_000362 |
| | | | | | | | | metalloproteinase 3 (Sorsby | |
| | | | | | | | | fundus dystrophy, | |
| | | | | | | | | pseudoinflammatory) | |
| P01086_F06 | 2.1 | −2.4 | −1 | 2.6 | −3.1 | −1.2 | TIMP3 | tissue inhibitor of | NM_000362 |
| | | | | | | | | metalloproteinase 3 (Sorsby | |
| | | | | | | | | fundus dystrophy, | |
| | | | | | | | | pseudoinflammatory) | |
| P01071_A06 | −3 | 3 | −1 | −2.5 | 2.6 | −1 | TM4SF1 | transmembrane 4 superfamily | NM_014220 |
| | | | | | | | | member 1 | |
| P01099_E08 | −1.6 | 1.8 | 1 | −1.8 | 1.5 | −1.1 | TncRNA | trophoblast-derived noncoding | |
| | | | | | | | | RNA | |
| P01126_E09 | −1.7 | 2 | 1 | −3.7 | 4.2 | 1.1 | TNFAIP2 | tumor necrosis factor, alpha- | NM_006291 |
| | | | | | | | | induced protein 2 | |
| P01085_A06 | −1.5 | 1.6 | −1 | −2.4 | 1.9 | 1.1 | TNFAIP3 | tumor necrosis factor, alpha- | NM_006290 |
| | | | | | | | | induced protein 3 | |
| P01138_G10 | 1.8 | −1.7 | 1.2 | 2.1 | −2 | 1 | TNFRSF12A | tumor necrosis factor receptor | NM_016639 |
| | | | | | | | | superfamily, member 12A | |
| P01078_E05 | −2.1 | 3 | 1.4 | −2.6 | 2.5 | 1.2 | TNFSF10 | tumor necrosis factor (ligand) | NM_003810 |
| | | | | | | | | superfamily, member 10 | |
| P01144_C11 | −2 | 2 | 1 | −1.9 | 1.4 | 1.2 | TOP2A | topoisomerase (DNA) II alpha | NM_001067 |
| | | | | | | | | 170 kDa | |
| P01140_D03 | 2.1 | −1.7 | −1 | 1.2 | −1.6 | −1 | TTID | titin immunoglobulin domain | NM_006790 |
| | | | | | | | | protein (myotilin) | |
| P01070_H07 | −2.9 | 2.3 | 1.1 | −3 | 2.6 | −1.1 | TXNRD1 | thioredoxin reductase 1 | NM_003330 |
| P01089_D01 | 1.7 | −3 | 1.1 | 2.5 | −2.3 | −1 | UCHL1 | ubiquitin carboxyl-terminal | NM_004181 |
| | | | | | | | | esterase L1 (ubiquitin | |
| | | | | | | | | thiolesterase) | |
| P01123_D07 | −1.9 | 2.4 | 1.1 | −2 | 1.8 | 1.1 | UGCG | UDP-glucose ceramide | NM_003358 |
| | | | | | | | | glucosyltransferase | |
| P01089_F07 | 1.5 | −2.6 | 1.2 | 2.4 | −1.7 | 1.2 | UMPK | undine monophosphate kinase | NM_012474 |
| P01070_F11 | 2.1 | −3.1 | 1.1 | 2.4 | −1.9 | 1 | UMPS | uridine monophosphate | NM_000373 |
| | | | | | | | | synthetase (orotate | |
| | | | | | | | | phosphoribosyl transferase | |
| | | | | | | | | and orotidine-5′- | |
| | | | | | | | | decarboxylase) | |
| P01061_B02 | −2.7 | 2.4 | 1 | −4.2 | 2.3 | −1.3 | VCAM1 | vascular cell adhesion | NM_001078 |
| | | | | | | | | molecule 1 | |
| P01141_C06 | 2.7 | −1.8 | 1.3 | 1.4 | −1.2 | 1.2 | WISP1 | WNT1 inducible signaling | NM_003882 |
| | | | | | | | | pathway protein 1 | |
| P00777_C09 | −1.6 | 1.8 | 1.1 | −5 | 4 | −1 | WISP2 | WNT1 inducible signaling | NM_003881 |
| | | | | | | | | pathway protein 2 | |
| P00777_C10 | −2.2 | 2.1 | 1.1 | −5.6 | 4.4 | −1 | WISP2 | WNT1 inducible signaling | NM_003881 |
| | | | | | | | | pathway protein 2 | |
| P01126_H07 | −1.8 | 1.6 | 1.1 | −3 | 3.9 | −1 | WISP2 | WNT1 inducible signaling | NM_003881 |
| | | | | | | | | pathway protein 2 | |
| P01142_D08 | 3.7 | −3 | 1.3 | 3.6 | −2.8 | 1.1 | XRCC4 | X-ray repair complementing | NM_003401 |
| | | | | | | | | defective repair in Chinese | |
| | | | | | | | | hamster cells 4 | |
| P01104_H07 | −1.7 | 1.7 | 1.2 | −1.9 | 1.5 | 1.1 | ZFPM2 | zinc finger protein, multitype 2 | NM_012082 |
| P01064_H12 | −1.4 | 1.5 | 1.1 | −1.8 | 1.5 | −1 | ZNF142 | zinc finger protein 142 (clone | NM_005081 |
| | | | | | | | | pHZ-49) | |
| P01075_E02 | 1.5 | −1.2 | 1.1 | 1.9 | −1.9 | 1 | ZNF193 | zinc finger protein 193 | NM_006299 |

Validation of Microarray by Real-Time RT-PCR and Western Blot Analyses

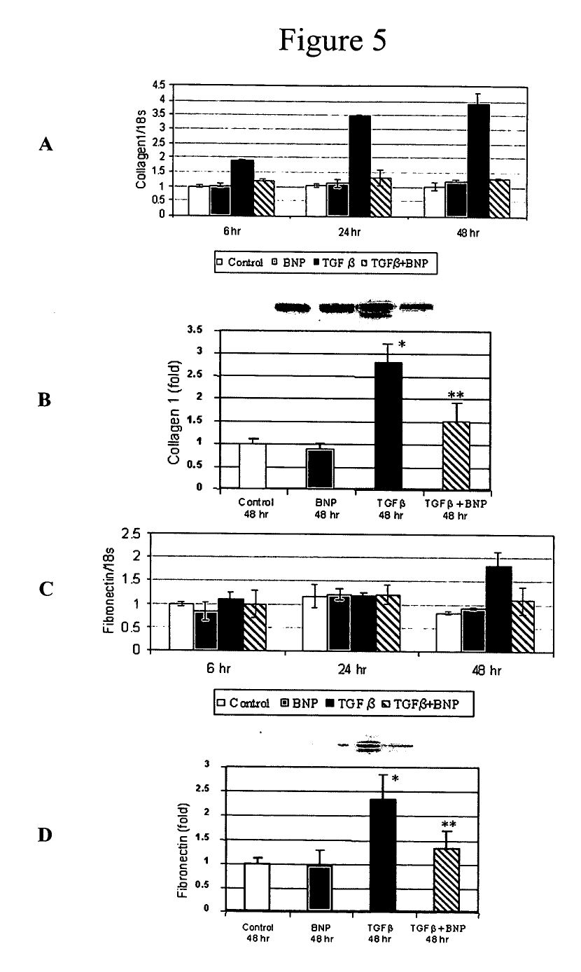

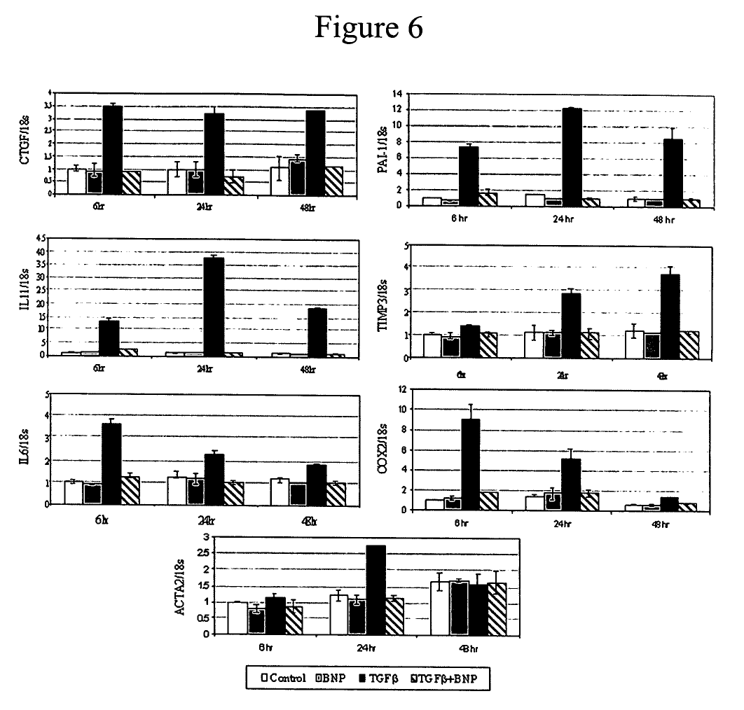

Representative microarray data was validated using real-time RT-PCR and Western analyses. TGFβ induced Collagen 1 mRNA levels in human cardiac fibroblasts at 6, 24, and 48 h; this induction was blocked by BNP at all 3 time points (FIG. 5A). Collagen 1 protein synthesis was also induced (˜3-fold) at 48 h, and BNP inhibited this stimulation by ˜75% (FIG. 5B). BNP also inhibited TGFβ-induced Fibronectin mRNA and protein expression at 48 h (FIG. 5C,D). These data corroborate the microarray results, with the exception of Fibronectin, which did not exceed the array differential expression threshold value, most likely due to the lower sensitivity of the microarray compared to real-time RT-PCR. The effects of BNP on TGFβ stimulation of pro-fibrotic genes CTGF, PAI-1, TIMP3, IL11, and ACTA2 were also confirmed by real-time RT-PCR (FIG. 6). Additional verification was obtained for the pro-inflammatory genes COX2 and IL6 at 6, 24, and 48 h (FIG. 6). Again, most likely due to sensitivity issues, IL6 was not included in FIG. 4C, since it did not exceed the array differential expression threshold value.

In addition, real-time RT-PCR assays were performed for 9 genes on primary cultures of human cardiac fibroblasts from a second independent donor lot of fibroblasts (see Table 3). The effects of BNP on TGFβ-induced gene expression in both donors were similar, although donor lot 2 was slightly less responsive to TGFβ. Taken together, these results confirm the microarray data using independent assay methods, as well as, multiple human cardiac fibroblast donors.

TABLE 3
Real-time RT-PCR validation of microarray data using human cardiac fibroblasts from
two separate donors (lot 1 and lot 2). Expression levels are normalized to 18s RNA and
are shown relative to the control samples. Standard deviations reflect duplicate
biological replicates; real-time RT-PCR reactions were performed in triplicate.

| Gene | Control | BNP | TGFβ | TGFβ + BNP | Time (h) | Lot |
|---|---|---|---|---|---|---|
| Collagen 1 | 1.0 ± 0.05 | 1.0 ± 0.05 | 1.9 ± 0.04 | 1.2 ± 0.01 | 6 | 1 |
| | 1.0 ± 0.06 | 1.1 ± 0.13 | 3.3 ± 0.05 | 1.3 ± 0.26 | 24 | 1 |
| | 1.0 ± 0.11 | 1.0 ± 0.26 | 1.5 ± 0.09 | 1.2 ± 0.01 | 24 | 2 |
| | 1.0 ± 0.13 | 1.2 ± 0.03 | 3.8 ± 0.38 | 1.3 ± 0.03 | 48 | 1 |
| | 1.0 ± 0.20 | 1.0 ± 0.01 | 2.5 ± 0.32 | 1.3 ± 0.18 | 48 | 2 |
| Fibronectin | 1.0 ± 0.04 | 0.9 ± 0.19 | 1.1 ± 0.17 | 1.0 ± 0.29 | 6 | 1 |
| | 1.0 ± 0.21 | 1.0 ± 0.10 | 1.0 ± 0.05 | 1.0 ± 0.18 | 24 | 1 |
| | 1.0 ± 0.19 | 0.9 ± 0.24 | 1.0 ± 0.02 | 1.0 ± 0.12 | 24 | 2 |
| | 1.0 ± 0.04 | 1.1 ± 0.04 | 2.2 ± 0.38 | 1.3 ± 0.35 | 48 | 1 |
| | 1.0 ± 0.01 | 1.0 ± 0.11 | 2.0 ± 0.39 | 1.5 ± 0.02 | 48 | 2 |
| SERPINE1/PAI-1 | 1.0 ± 0.07 | 0.7 ± 0.08 | 7.3 ± 0.44 | 1.7 ± 0.37 | 6 | 1 |
| | 1.0 ± 0.01 | 0.7 ± 0.01 | 8.5 ± 0.08 | 0.7 ± 0.10 | 24 | 1 |
| | 1.0 ± 0.10 | 0.7 ± 0.11 | 2.4 ± 0.06 | 1.1 ± 0.10 | 24 | 2 |
| | 1.0 ± 0.22 | 0.9 ± 0.00 | 8.4 ± 1.33 | 0.9 ± 0.13 | 48 | 1 |
| | 1.0 ± 0.17 | 0.8 ± 0.03 | 2.6 ± 0.03 | 0.9 ± 0.06 | 48 | 2 |
| CTGF | 1.0 ± 0.15 | 0.9 ± 0.24 | 3.5 ± 0.08 | 0.9 ± 0.03 | 6 | 1 |
| | 1.0 ± 0.28 | 1.0 ± 0.29 | 3.3 ± 0.25 | 0.7 ± 0.25 | 24 | 1 |
| | 1.0 ± 0.09 | 1.5 ± 0.44 | 2.2 ± 0.16 | 1.5 ± 0.04 | 24 | 2 |
| | 1.0 ± 0.45 | 1.4 ± 0.13 | 3.1 ± 0.01 | 1.1 ± 0.01 | 48 | 1 |
| | 1.0 ± 0.32 | 1.3 ± 0.12 | 2.1 ± 0.14 | 1.0 ± 0.24 | 48 | 2 |
| IL11 | 1.0 ± 0.20 | 1.1 ± 0.04 | 13.3 ± 0.89 | 2.1 ± 0.06 | 6 | 1 |
| | 1.0 ± 0.13 | 1.2 ± 0.07 | 32.3 ± 0.82 | 1.1 ± 0.14 | 24 | 1 |
| | 1.0 ± 0.06 | 1.0 ± 0.05 | 7.7 ± 0.81 | 2.1 ± 0.18 | 24 | 2 |
| | 1.0 ± 0.23 | 0.7 ± 0.10 | 17.6 ± 0.22 | 1.0 ± 0.08 | 48 | 1 |
| | 1.0 ± 0.09 | 0.8 ± 0.09 | 5.9 ± 0.18 | 1.2 ± 0.10 | 48 | 2 |
| TIMP3 | 1.0 ± 0.01 | 0.9 ± 0.11 | 1.4 ± 0.03 | 1.0 ± 0.12 | 6 | 1 |
| | 1.0 ± 0.31 | 1.0 ± 0.12 | 2.6 ± 0.26 | 1.0 ± 0.23 | 24 | 1 |
| | 1.0 ± 0.13 | 0.7 ± 0.09 | 1.5 ± 0.12 | 1.3 ± 0.14 | 24 | 2 |
| | 1.0 ± 0.26 | 0.9 ± 0.00 | 3.0 ± 0.34 | 1.0 ± 0.09 | 48 | 1 |
| | 1.0 ± 0.08 | 0.6 ± 0.00 | 1.7 ± 0.13 | 0.8 ± 0.01 | 48 | 2 |
| IL6 | 1.0 ± 0.06 | 0.9 ± 0.02 | 3.6 ± 0.27 | 1.3 ± 0.14 | 6 | 1 |
| | 1.0 ± 0.13 | 0.9 ± 0.21 | 1.7 ± 0.14 | 0.8 ± 0.03 | 24 | 1 |
| | 1.0 ± 0.09 | 0.9 ± 0.07 | 1.4 ± 0.05 | 1.0 ± 0.11 | 24 | 2 |
| | 1.0 ± 0.13 | 0.9 ± 0.03 | 1.6 ± 0.12 | 0.9 ± 0.05 | 48 | 1 |
| | 1.0 ± 0.17 | 0.9 ± 0.06 | 1.4 ± 0.17 | 0.9 ± 0.17 | 48 | 2 |
| PTGS2/COX-2 | 1.0 ± 0.01 | 1.2 ± 0.22 | 9.0 ± 1.49 | 1.8 ± 0.05 | 6 | 1 |
| | 1.0 ± 0.08 | 1.2 ± 0.38 | 3.5 ± 0.67 | 1.2 ± 0.19 | 24 | 1 |
| | 1.0 ± 0.07 | 1.1 ± 0.05 | 4.9 ± 0.36 | 1.4 ± 0.18 | 24 | 2 |
| | 1.0 ± 0.10 | 1.0 ± 0.12 | 2.2 ± 0.12 | 1.3 ± 0.03 | 48 | 1 |
| | 1.0 ± 0.19 | 1.0 ± 0.06 | 5.4 ± 0.92 | 1.2 ± 0.01 | 48 | 2 |
| ACTA2 | 1.0 ± 0.03 | 0.8 ± 0.12 | 1.1 ± 0.11 | 0.9 ± 0.20 | 6 | 1 |
| | 1.0 ± 0.14 | 0.9 ± 0.11 | 2.2 ± 0.00 | 0.9 ± 0.07 | 24 | 1 |
| | 1.0 ± 0.04 | 0.9 ± 0.25 | 2.3 ± 0.12 | 1.6 ± 0.41 | 24 | 2 |
| | 1.0 ± 0.17 | 1.0 ± 0.03 | 1.0 ± 0.19 | 1.0 ± 0.21 | 48 | 1 |
| | 1.0 ± 0.05 | 0.7 ± 0.11 | 2.5 ± 0.13 | 1.0 ± 0.12 | 48 | 2 |

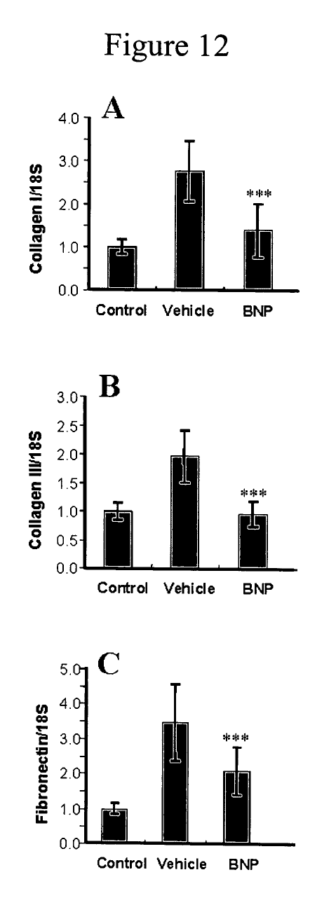

In a related study, a gene microassay profile of rat heart tissue was conducted. The results of this study are shown in FIG. 12. Fibrotic and extracellular matrix associated genes were stimulated in vivo by L-NAME plus angiotensin II. MRNA expression for collagen I, collagen III, and fibronectin was markedly reduced by the administration of BNP.

MEK/ERK Pathway Involved in BNP's Anti-Fibrotic Role

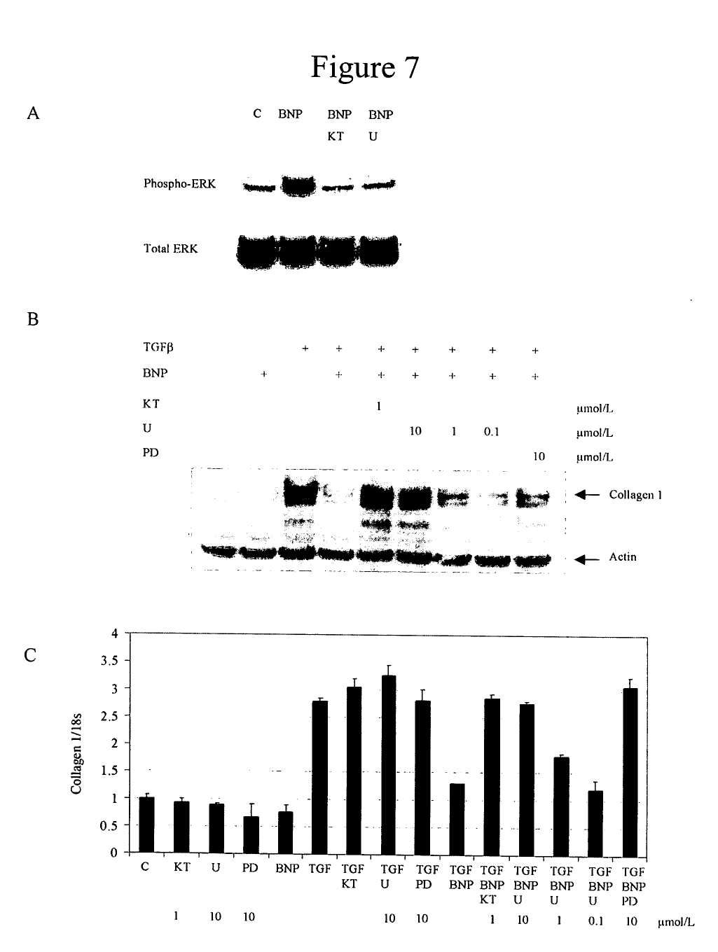

Natriuretic peptides were previously shown to stimulate ERK activity in cardiac myocytes and vascular endothelial cells. The MEK/ERK pathway has been linked to the repression of TGFβ/Smad signaling. To determine whether PKG or ERK signaling is involved in BNP-dependent attenuation of TGFβ signaling, cultured cells were treated with BNP and/or TGFβ in the presence of a PKG inhibitor (KT5823) or two different MEK inhibitors (U0126, PD98059). BNP induced ERK phosphorylation was completely blocked by KT5823 and U0126, indicating that BNP activates ERK via PKG and MEK signaling cascades (FIG. 7a). Both MEK inhibitors (U0126, PD98059) reversed BNP inhibition of TGFβ-induced Collagen-1 expression analyzed by Western blot (FIG. 7b) and real-time RT-PCR (FIG. 7c). A similar result was demonstrated for PAI-1 using real-time RT-PCR. These findings suggest that the ERK pathway plays an important role in BNP-dependent inhibition of the fibrotic response induced by TGFβ in human cardiac fibroblasts.

Fibrosis and ECM

One of the key features of cardiac fibrosis is the increased deposition of the ECM. The dynamic turnover of ECM proteins is controlled by several regulatory mechanisms: de novo biosynthesis of ECM components, proteolytic degradation of ECMs by matrix metalloproteinases (MMPs), and inhibition of MMP activities by endogenous inhibitors, TIMPs. All of these processes have been shown to be profoundly affected by TGFβ. The results provided herein suggest that TGFβ-induced ECM deposition in human cardiac fibroblasts occurs largely by increasing ECM gene expression, including Fibronectin, COL1A2, COL15A, COL7A1, MAGP2, MATN3, FBN1, and COMP. Fibronectin and collagen expression in cardiac fibroblasts has been well-established in the fibrotic response, however, this is the first report of TGFβ induction of other ECM genes including MAGP2, MATN3, FBN1 and COMP, further corroborating TGFβ's role in ECM induction. Interestingly, COMP, which is a member of the thrombospondin family, has been shown to have a direct interaction with Fibronectin,²⁵ supporting its role in fibrotic processes. We also found Thombospondin 2, which is involved in the activation of latent TGFβ²⁶ regulated by TGFβ in our studies and opposed by BNP (Table 2). Also sharing close identity with the latent TGFβ family of binding proteins is FBN1, a component of extracellular microfibrils. The opposing effects of BNP on these gene regulatory events, suggests that BNP modulates cardiac fibrosis.

In addition to the suppression of TGFβ-induced ECM biosynthesis, BNP may also modulate the degradation of ECM proteins by opposing elevated TIMP3 levels in TGFβ-stimulated cells. The TIMP family of proteins is believed to play significant roles in controlling extracellular matrix remodeling. Elevation of TIMP3 expression has been observed in animal models of myocardial infarction, suggesting that it may be a contributor to matrix remodeling in the failing heart.

Another hallmark of the fibrotic process is the transformation of cardiac fibroblasts to myofibroblasts and the induction of pro-fibrotic mediators. Myofibroblasts acquire contractile properties similar to smooth muscle cells. The results provided above demonstrate that BNP inhibited TGFβ-induction of several myofibroblast markers including ACTA2 and MYH9. BNP also inhibited TGFβ pro-fibrotic mediators, such as, CTGF, PAI-1, and IL11. CTGF and PAI-1 are well-established downstream signaling genes of the TGFβ pathway, and IL11 has been associated with tissue remodeling and fibrosis. IL11 expression in cardiac fibroblasts also seems to contribute to TGFβ-mediated fibrosis. The use of BNP to suppress this response should result in a protective effect.

Collectively, these effects of BNP on gene expression in TGFβ-stimulated cells demonstrate a role for BNP in anti-fibrotic processes in cardiac fibroblasts. In striking contrast to TGFβ-treated cells, BNP had no significant effects in unstimulated fibroblasts. This is consistent with the physiological actions of BNP, working only in opposition to other hormonal systems such as the renin-angiotensin-aldosterone system.

Changes in Cell Proliferation

The effects of TGFβ on cell growth is cell-type dependent. As provided above, TGFβ stimulated cardiac fibroblast proliferation. Whether TGFβ has a direct effect on cell cycle or an indirect effect through other mechanisms is unclear. However, cDNA microarray analysis revealed that BNP markedly inhibits the expression of a number of TGFβ-induced growth factors or growth factor-like genes including PDGFA, IGF1, FGF18, and IGFBP10 (CYR61). The up-regulation of these genes by TGFβ could partially explain the induction of cell proliferation, suggesting that it may be mediated indirectly through the stimulation of growth factor productions. TGFβ also induced the expression of PTHLH (PTHrP), which has known chronotropic and vasodilatory effects. In osteoblast-like cells PTHrP can induce cell proliferation. Interestingly, in the myocardium, PTHrP levels are increased in congestive heart failure (CHF).

The growth inhibitory effects of natriuretic peptides have previously been reported. Cao and Gardner first demonstrated that natriuretic peptides inhibit PDGF, FGF2, and mechanical stretch-induced DNA synthesis in neonatal rat cardiac fibroblasts. Consistent with these findings, natriuretic peptides and cyclic GMP have been reported to inhibit cell proliferation induced by angiontensin II, endothelin-1, and norepinephrine in many cell types including cardiac fibroblasts, vascular smooth muscle cells, endothelial cells, and mesangial cells. The results provided herein suggest an important role for BNP in regulating fibroblast growth during cardiac remodeling.

Changes in Inflammatory Genes

Cardiac expression of cytokines is thought to contribute to a decrease in left ventricle contractile performance and deleterious remodeling. Although similar effects have been observed with ANP, reported herein for the first time is that brain natriuretic peptide blocks TGFβ stimulation of several pro-inflammatory genes including COX2, IL6, TNFAIP6, and TNFSF4.

TGFβ has a dual effect in the regulation of inflammatory processes. For example, it increases COX2 expression and prostaglandin E2 release in pulmonary artery smooth muscle cells, airway smooth muscle cells, and intestinal epithelial cells. On the other hand, TGFβ down-regulates the production of MCP-1 and complement components (C3 and C4) in human proximal tubular epithelial cells and macrophages. The results provided herein corroborates the dual effect of TGFβ in the modulation of inflammatory gene expression in cardiac fibroblasts. From these results, it was found that while TGFβ induced some inflammatory genes, it down-regulated others, such as, IL1b, MCP1-R, GRO1, GRO3, and MCP4. Both effects are reversed by BNP. However, in the absence of TGFβ stimulation, BNP had no significant effect on the expression of inflammatory genes. It is likely that a balance of pro- and anti-inflammatory stimuli is important in the process of cardiac remodeling.

Signaling Mechanism Underlying BNP's Anti-Fibrotic Role

Studies aimed at elucidating the mechanism of BNP's inhibition of a fibrotic response indicate that the ERK signaling pathway plays an important role. The results provided herein demonstrate that BNP phosphorylates ERK via PKG-dependent signaling in primary human cardiac fibroblasts. Moreover, this activation attenuates the TGFβ-induced fibrotic response as measured by Collagen 1 expression. This is consistent with previous studies showing that ERK activation is required for both the anti-hypertrophic effect of ANP in cardiac myocytes, and the inhibition of TGFβ signaling in mammary and lung epithelial cells.

In Vivo Studies

In a related study, an in vivo model for acute myocardial injury was used to explore the effects of BNP. Male Sprague Dawley rats ranging in weight from 225 to 250 gm were utilized. Acute myocardial injury was induced by administration of Nω-nitro-L-arginine methyl ester (L-NAME, 40 mg/kg/day)salt (1% NaCl) plus angiotensin II (AngII, 0.5 mg/kg/day) in the rats. The L-NAME was administered in drinking water from day 1 to day 14. Angiotensin II was continuously infused subcutaneously with an osmotic pump from day 11 to day 14. Rat BNP (400 mg/kg/min) was intravenously infused through an external infusion pump from day 10 to day 14.

Systolic blood pressure, plasma level of aldosterone, cardiac function heart/body weight ration and gene expression in the heart were analyzed. Systolic blood pressure was monitored via tail cuff technique with an IITC blood pressure recording system. Cardiac function was monitored via a Millar ARIA Pressure Volume Conductance System with an 1.4 F catheter. Gene expression as referenced above with results provided in FIG. 12 were monitored by RT-PCR with an ABI Prism™ 7700 sequence detection system.

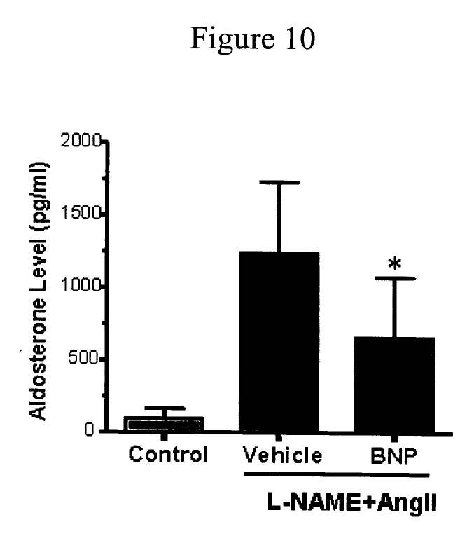

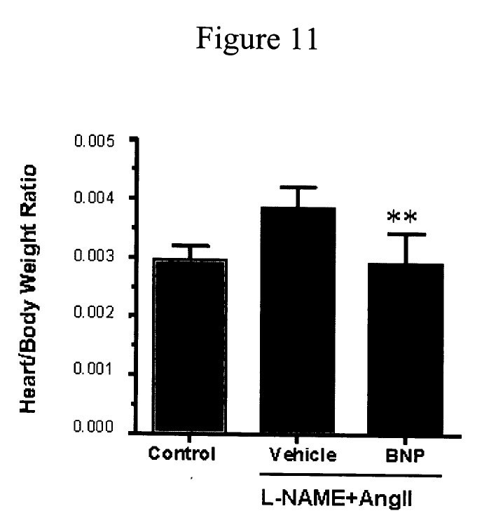

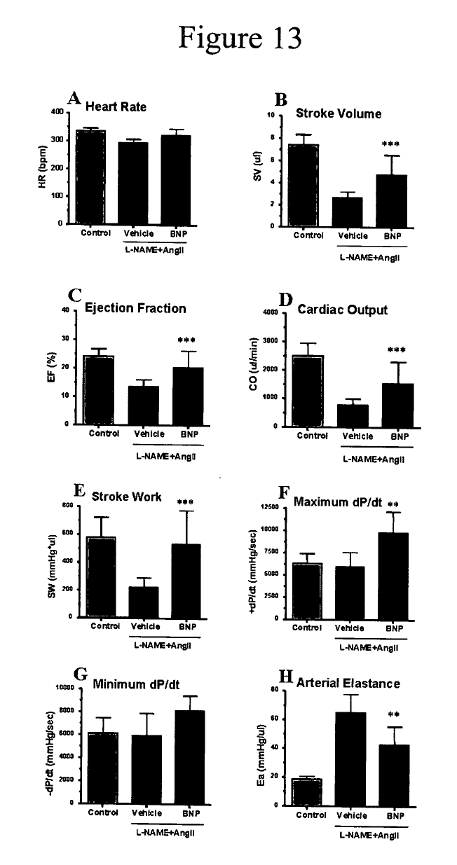

It was observed that BNP had no effect on systolic blood pressure raised by L-NAME+AngII but significantly attenuated aldosterone1.25.2±0.2 vs. 6.6±0.16 ng/ml, p<0.05). See FIG. 10. As shown in FIG. 13, BNP improved cardiac function by significantly increase in stroke volume (2.68±0.23 vs. 4.74±0.73 ul, p<0.05), ejection fraction (13.6±1.1 vs. 20.4±2.4% p<0.05), and diastolic volume (19.0±0.9 vs 22.4±1.1 ul, p<0.05) and stroke work (223.0±29.4 vs 531.5±99.1 mmH*ul, p<0.05), and decrease in arterial elastance (6.50±5.7 vs 42.6±5.1 mmHg/ul, p<0.01). As shown in FIG. 11, BNP significantly reduced the heart/body weigh ratio (0.0039±0.002 vs. 0.0029±0.001, p<0.05) and as referenced above, abolished the profibrotic phenotype indicated by decreasing expression of collagen I (p<0.01), collagen III (p<0.05) and fibronectin (p<0.05).

### SUMMARY

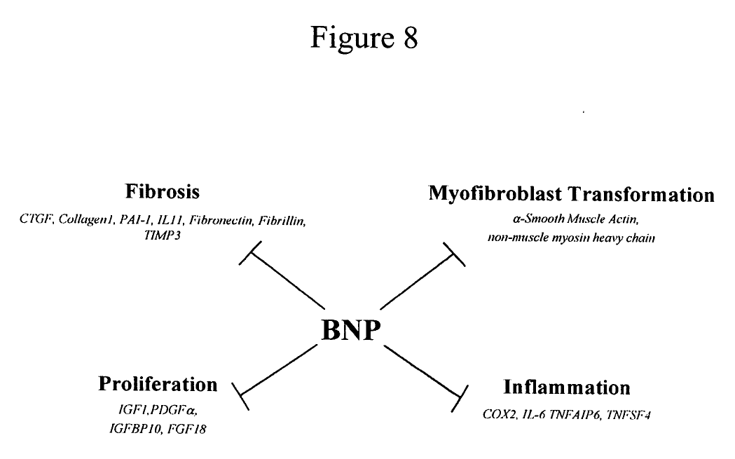

Along with the endothelin pathway, the renin-angiotensin and aldosterone system, the fibrosis-promoting TGFβ pathway is important in the pathophysiology of heart failure. BNP appears to oppose TGFβ-regulated gene expression related to fibrosis and myofibroblast conversion. Furthermore, BNP's opposition to the TGFβ-stimulated fibrotic response is dependent on the PKG and the MEK/ERK pathways. This finding is consistent with the observation that BNP deficient mice show increased fibrosis and Collagen 1 expression. In addition to BNP's global effects on fibrosis, it may also have effects on other processes, such as inflammation and proliferation (FIG. 8). These findings support a beneficial role for BNP in the prevention of cardiac fibrosis and the treatment of cardiac diseases. They also provide the first demonstration that BNP has a direct effect on cardiac fibroblasts to oppose a TGFβ-induced fibrotic response, suggesting that BNP functions as an anti-fibrotic factor in the heart to prevent cardiac remodeling in pathological conditions.

Independent from the antifibrotic effect, the in vivo studies as provided herein indicate that BNP may be used to reduce cardiac remodeling and prevent subsequent heart failure. BNP may also be useful as a cardioprotective agent to improve cardiac function post acute myocardial injury such as myocardial infarction.

All references cited throughout the specification are expressly incorporated herein by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, and the like. All such modifications are within the scope of the claims appended hereto.

What is claimed is:

1. A method for treating cardiac remodeling in a subject that has undergone myocardial injury, said method comprising administering a therapeutically effective amount of natriuretic peptide to said subject.

2. A method for treating cardiac dysfunction in a subject that has undergone myocardial injury, said method comprising administering a therapeutically effective amount of natriuretic peptide to said subject.

3. A method for treating cardiac fibrosis in a subject who has undergone myocardial injury, said method comprising administering a therapeutically effective amount of natriuretic peptide to said subject.

4. The method of claims 1 or 2 wherein said natriuretic peptide is brain natriuretic peptide.

5. A method of inhibiting the production of Collagen 1, Collagen 3 or Fibronectin protein in a subject who has undergone myocardial injury, said method comprising administering a therapeutically effective amount of brain natriuretic peptide to said subject.

6. A method of alleviating or reversing the effect of TGFβ mediated cell activation in cardiac tissue on the expression of one or more genes associated with fibrosis, comprising contacting one or more cells or tissues in which the expression of said genes is altered as a result of TGFβ mediated activation, with brain natriuretic peptide.

7. The method of claim 5 wherein said genes are selected from the group consisting essentially of Collagen1, Collagen 3, Fibronectin, CTGF, PAI-1, and TIMP3.

8. A method of inhibiting the transformation of cardiac fibroblast cells into myofibroblast cells in a subject that has undergone myocardial injury, said method comprising administering a therapeutically effective amount of brain natriuretic peptide to said subject.
